(19) United States
(12) Patent Application Publication
(10) Pub. No.: US 2026/0098081 A1
(43) Pub. Date: Apr. 9, 2026

(54) METABOLIC SELECTION VIA THE GLYCINE-FORMATE BIOSYNTHESIS PATHWAY

(71) Applicant: Sigma-Aldrich Co. LLC., St. Louis, MO (US)

(72) Inventors: Samuel Peters, St. Louis, MO (US); James Ravellette, St. Charles, MO (US); David Razafsky, Olivette, MO (US); Jason A. Gustin, Richmond Heights, MO (US); Trissa Borgschulte, St. Charles, MO (US)

(21) Appl. No.: 19/112,697

(22) PCT Filed: Sep. 29, 2023
(86) PCT No.: PCT/US2023/075554

§ 371 Date: Mar. 18, 2025

Related U.S. Application Data
(60) Provisional application No. 63/377,874, filed on Sep. 30, 2022.

(51) Int. Cl. C07K 16/00; C07K 1/14; C12N 5/071; C12N 5/10; C12N 9/10; C12N 9/22

(52) U.S. Cl. CPC C07K 16/00; C07K 1/14; C12N 5/0682; C12N 5/10; C12N 9/1014; C12N 9/222; C12Y 201/02001; C07K 2317/31; C12N 2510/02; C12N 2510/04

(57) ABSTRACT

The present disclosure provides an isolated mammalian cell comprising a reduced or eliminated expression of Serine Hydroxymethyltransferase 2 (SHMT2). Further provided are methods for preparing such cells and methods for using such cells for the production of recombinant proteins.

### CROSS-REFERENCE TO RELATED APPLICATIONS

The present application claims the benefit of priority of U.S. Provisional Application No. 63/377,874, filed Sep. 30, 2022, the entire content of which is incorporated herein by reference.

### FIELD

This disclosure relates to mammalian cell lines for use in biologic production systems, wherein the mammalian cell lines are engineered to have reduced or eliminated expression of a component of the glycine-formate biosynthesis pathway to create a glycine auxotrophic cell line.

### BACKGROUND

Development of high producing clonal cell lines for biomanufacturing typically utilizes one or more well-known selection methods, such as glutamine synthetase (GS for glutamine selection), dihydrofolate receptor (DHFR for hypoxathine and thymidine selection), antibiotic selection (puromycin, hygromycin, blasticidin, etc.), or P5C Synthetase (P5CS—proline selection). The GS system has become a standard in the industry, but there is a need for cell lines that permit multiple selection methods so that more than one vector can be introduced into the cell line to facilitate production of molecules such as bispecific antibodies, multispecific antibodies, and other multichain enzymes/proteins or proteins/enzymes that require an effector protein for expression.

### SUMMARY

Among the various aspects of the present disclosure is the provision of mammalian cell lines for use in biologic production systems, wherein the mammalian cell lines are engineered to have reduced or eliminated expression of the endogenous Serine hydroxymethyl transferase 2 (SHMT2) gene. In the absence of endogenously expressed functional SHMT2 protein the cells require an exogenous source of the amino acid glycine and/or a single carbon source, for example formate to survive and/or grow. The chromosomal SHMT2 sequence can be inactivated using targeting endonuclease-mediated genome modification, e.g., CRISPR ribonucleoprotein (RNP) complexes or zinc finger nucleases. In other aspects of the present disclosure is the provision of mammalian cell lines, wherein the mammalian cell lines are engineered to have reduced or eliminated expression of the endogenous SHMT2 gene and reduced or eliminated expression of the endogenous Glutamine Synthetase (GS) gene.

Another aspect of the present disclosure encompasses processes for selecting cell lines that have enhanced productivity of expressed biotherapeutic proteins. In other aspects of the present disclosure is the provision of a bioproduction system for expression of bispecific antibodies or biotherapeutic proteins that require expression of effector proteins more conveniently by utilizing multiple selection systems. The processes comprise expressing at least one recombinant protein in any of the mammalian cell lines.

Other aspects and iterations of the disclosure are described in more detail below.

### BRIEF DESCRIPTION OF THE DRAWINGS

FIG. 1 Serine hydroxymethyltransferase 2 (Shmt2) converts serine to glycine in the mitochondria.

FIG. 2 Shmt2 cDNA sequence in CHO. The gRNA target site is underlined and the NGG PAM is in bold.

FIG. 3. A) Genotypes of Shmt2 KO clones were confirmed with NGS. Insertions or deletions and their respective frequencies are in bold. B) KO alleles generated by CRISPR-Cas9 targeting. All base pair modifications above produce an early stop codon in the coding sequence.

FIG. 4 Vectors were designed to allow for selection of GFP positive cells using a glutamine-based selection system and CFP positive cells using a glycine-formate based selection system as well as development of secreted recombinant proteins via the use of two similar vectors that contain a mAb heavy chain, light chain, and either the Shmt2 or GS coding sequence.

FIG. 5 Shmt2 KO clones 1B4, 5D5, and 8F9 are unable to survive without glycine while the parental cell line GS−/− Shmt2+/+ survives in the presence or absence of glycine.

FIG. 6 CHO cells with genetically disrupted GS and Shmt2 genes transfected with a plasmid containing GS+GFP expression cassettes were selected in glutamine deficient media and express GFP, but do not express CFP (left). CHO cells with genetically disrupted GS and Shmt2 genes transfected with a plasmid containing Shmt2+CFP expression cassettes were selected in glycine deficient media and express CFP, but do not express GFP (middle). CHO cells with genetically disrupted GS and Shmt2 genes co-transfected with Shmt2+CFP and GS+GFP were selected in media deficient of both glycine and glutamine and express high levels of both GFP and CFP (right).

FIG. 7 Copy number of endogenous Shmt2 in the GS−/− Shmt2+/+ cell line via ddPCR.

FIG. 8 Viability and growth of pools expressing GFP, CFP or GFP and CFP using GS, Shmt2, or GS and Shmt2 (Dual), selection respectively.

FIG. 9 Sodium formate supplementation in the media increases the growth rate of the Shmt2 KO clones in a dose dependent manner. Supplementation of 200 uM sodium formate leads to a growth rate comparable to SHMT2^{+/+} cells.

FIG. 10 Shmt2 KO cells are unable to survive without glycine even in the presence of sodium formate.

FIG. 11 The dual expressing, bulk pool showed the highest viability and highest viable cell density in a fed batch assay.

FIG. 12 All bulk pools showed production of mAb with the dual expressing pool (GS-SO57+Shmt2-SO57) showing the highest protein production (left panel). GS-SO57 and Shmt2-SO57 showed lower levels of protein production (right panel).

### DETAILED DESCRIPTION

The present disclosure provides mammalian cell lines engineered to have reduced or eliminated expression of the endogenous SHMT2 gene. Further provided are mammalian cell lines engineered to have reduced or eliminated expression of the endogenous GS gene and reduced or eliminated expression of the endogenous SHMT2 gene. Methods for producing said engineered cell lines are provided, as well as methods of selecting and using said engineered cell lines to produce recombinant proteins.

### (I) Engineered Cell Lines

One aspect of the present disclosure encompasses mammalian cell lines that are engineered to have reduced or eliminated expression of the endogenous SHMT2 gene. Alternatively, the mammalian cell lines are engineered to have reduced or eliminated expression of both the endogenous SHMT2 gene and the endogenous GS gene.

The cell lines disclosed herein having reduced or eliminated expression of SHMT2 or reduced expression of SHMT2 and GS are genetically engineered to modify the chromosomal sequence encoding the SHMT2 or GS protein. Chromosomal sequences can be modified using targeted endonuclease-mediated genomic editing techniques, which are detailed below in section (III). For example, chromosomal sequences can be modified to comprise a deletion of at least one nucleotide, an insertion of at least one nucleotide, a substitution of at least one nucleotide, or a combination thereof, such that the reading frame is shifted and no protein product is produced or a non-functional protein is produced (i.e., the chromosomal sequence is inactivated). Inactivation of one allele of the chromosomal sequence encoding either SHMT2 or GS results in reduced expression (i.e., knock down) of the protein. Inactivation of both alleles of the chromosomal sequence encoding either SHMT2 or GS results in no expression (i.e., knock out) of the protein.

In some embodiments, the level of expression of SHMT2 can be reduced by at least about 5%, by at least about 10%, by at least about 20%, by at least about 30%, by at least about 40%, by at least about 50%, by at least about 60%, by at least about 70%, by at least about 80%, by at least about 90%, by at least about 95%, by at least about 99%, or more than about 99%. In other embodiments, the level of expression of SHMT2 can be reduced to non-detectable levels using techniques standard in the field (e.g., Western immunoblotting assays, ELISA enzyme assays, SDS polyacrylamide gel electrophoresis, and the like).

In general, cell viability, viable cell density, titer, growth rate, proliferation responses, cell morphology, apoptosis and autophagy levels, and/or general cell health of the engineered cell lines disclosed herein are similar to those of their non-engineered parental cells when supplemented with glycine, formate, a one carbon source and/or an exogenous SHMT2 coding sequence.

### (a) Cell Types

The engineered cell lines disclosed herein are mammalian cell lines. In some embodiments, the engineered cell lines can be derived from human cell lines. Non-limiting examples of suitable human cell lines includes human embryonic kidney cells (HEK293, HEK293T); human connective tissue cells (HT-1080); human cervical carcinoma cells (HELA); human embryonic retinal cells (PER.C6); human kidney cells (HKB-11); human liver cells (Huh-7); human lung cells (W138); human liver cells (Hep G2); human U2-OS osteosarcoma cells, human A549 lung cells, human A-431 epidermal cells, CACO-2 human colorectal adenocarcinoma cells, human pluripotent stem cells, Jurkat human T lymphocyte cells, or human K562 bone marrow cells. In other embodiments, the engineered cell lines can be derived from non-human cell lines. Suitable cell lines also include Chinese hamster ovary (CHO) cells; baby hamster kidney (BHK) cells; mouse myeloma NS0 cells; mouse myeloma Sp2/0 cell; mouse mammary gland C127 cells; mouse embryonic fibroblast 3T3 cells (NIH3T3); mouse B lymphoma A20 cells; mouse melanoma B16 cells; mouse myoblast C2C12 cells; mouse embryonic mesenchymal C3H-10T1/2 cells; mouse carcinoma CT26 cells, mouse prostate DuCuP cells; mouse breast EMT6 cells; mouse hepatoma Hepa1c1c7 cells; mouse myeloma J5582 cells; mouse epithelial MTD-1A cells; mouse myocardial MyEnd cells; mouse renal RenCa cells; mouse pancreatic RIN-5F cells; mouse melanoma X64 cells; mouse lymphoma YAC-1 cells; rat glioblastoma 9L cells; rat B lymphoma RBL cells; rat neuroblastoma B35 cells; rat hepatoma cells (HTC); buffalo rat liver BRL 3A cells; canine kidney cells (MDCK); canine mammary (CMT) cells; rat osteosarcoma D17 cells; rat monocyte/macrophage DH82 cells; monkey kidney SV-40 transformed fibroblast (COS7) cells; monkey kidney CVI-76 cells; or African green monkey kidney (VERO, VERO-76) cells. An extensive list of mammalian cell lines may be found in the American Type Culture Collection catalog (ATCC, Manassas, VA). In some embodiments, the cell lines disclosed herein are other than mouse cell lines. In certain embodiments, the engineered cell lines are CHO cell lines. Suitable CHO cell lines include, but are not limited to, CHO-K1, CHO-K1SV, CHO GS−/−, CHO S, DG44, DuxB11, and derivatives thereof.

In various embodiments, the parental cell lines can be deficient in glutamine synthase (GS), dihydrofolate reductase (DHFR), hypoxanthine-guanine phosphoribosyltransferase (HPRT), asparagine synthetase (ASNS), phosphoserine phosphatase (PSPH) or a combination thereof. For example, the chromosomal sequences encoding GS, DHFR, HPRT, ASNS and/or PSPH can be inactivated. In specific embodiments, all chromosomal sequences encoding GS, DHFR, HPRT, ASNS and/or PSPH are inactivated in the parental cell lines.

### (b) Optional Nucleic Acid Encoding Recombinant Protein

In some embodiments, the engineered cell lines disclosed herein can further comprise at least one nucleic acid encoding a recombinant protein. In general, the recombinant protein is heterologous, meaning that the protein is not native to the cell. The recombinant protein may be, without limit, a therapeutic protein chosen from an antibody, a fragment of an antibody, a monoclonal antibody, a humanized antibody, a humanized monoclonal antibody, a chimeric antibody, an IgG molecule, an IgG heavy chain, an IgG light chain, an IgA molecule, an IgD molecule, an IgE molecule, an IgM molecule, a vaccine, a growth factor, a cytokine, an interferon, an interleukin, a hormone, a clotting (or coagulation) factor, a blood component, an enzyme, a therapeutic protein, a nutraceutical protein, a functional fragment or functional variant of any of the forgoing, or a fusion protein comprising any of the foregoing proteins and/or functional fragments or variants thereof. In particular embodiments, the recombinant protein is a bispecific antibody or a multispecific antibody, or a protein that requires an effector protein for expression.

In some embodiments, the nucleic acid encoding the recombinant protein can be linked to sequence encoding serine hydroxymethyl transferase 2 (SHMT2), phosphoserine phosphatase (PSPH), asparagine synthetase (ASNS), hypoxanthine-guanine phosphoribosyltransferase (HPRT), dihydrofolate reductase (DHFR), and/or glutamine synthase (GS), such that SHMT2, PSPH, ASNS, HPRT, DHFR, and/or GS may be used as a selectable marker. The nucleic acid encoding the recombinant protein also can be linked to a sequence encoding at least one antibiotic resistance gene and/or sequence encoding marker proteins such as fluorescent proteins. In some embodiments, the nucleic acid encoding the recombinant protein can be part of an expression construct. The expression constructs or vectors can comprise additional expression control sequences (e.g., enhancer sequences, Kozak sequences, polyadenylation sequences, transcriptional termination sequences, etc.), selectable marker sequences, origins of replication, and the like. Additional information can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, NY, 3rd edition, 2001.

In some embodiments, the nucleic acid encoding the recombinant protein can be located extrachromosomally. That is, the nucleic acid encoding the recombinant protein can be transiently expressed from a plasmid, a cosmid, an artificial chromosome, a minichromosome, or another extrachromosomal construct. In other embodiments, the nucleic acid encoding the recombinant protein can be chromosomally integrated into the genome of the cell. The integration can be random or targeted. Accordingly, the recombinant protein can be stably expressed. In some iterations of this embodiment, the nucleic acid sequence encoding the recombinant protein can be operably linked to an appropriate heterologous expression control sequence (i.e., promoter). In other iterations, the nucleic acid sequence encoding the recombinant protein can be placed under control of an endogenous expression control sequence. The nucleic acid sequence encoding the recombinant protein can be integrated into the genome of the cell line using homologous recombination, targeting endonuclease-mediated genome editing, viral vectors, transposons, recombinase mediated cassette exchange systems, plasmids, and other well-known means. Additional guidance can be found in Ausubel et al. 2003, supra and Sambrook & Russell, 2001, supra.

### (II) Kits

A further aspect of the present disclosure provides kits for the production of recombinant proteins, wherein a kit comprises any of the engineered cell lines detailed above in section (I). A kit can further comprise cell growth media, transfection reagents, a plasmid vector, selection media, recombinant protein purification means, buffers, and the like. The kits provided herein generally include instructions for growing the cell lines and using them to produce recombinant proteins. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### (III) Methods for Preparing Engineered Cell Lines

Yet another aspect of the present disclosure provides methods for preparing or engineering the cell lines having reduced or eliminated expression of SHMT2 and/or GS, which are described above in section (I). Chromosomal sequences encoding SHMT2 and/or GS can be knocked-down or knocked-out using a variety of techniques. In general, the engineered cell lines are prepared using a targeting endonuclease-mediated genome modification process. Persons skilled in the art understand that said engineered cell lines also can be prepared using site-specific recombination systems, random mutagenesis, or other methods known in the art.

In general, engineered cell lines are prepared by a method comprising introducing into a parental cell line of interest at least one targeting endonuclease or nucleic acid encoding said targeting endonuclease, wherein the targeting endonuclease is targeted to a chromosomal sequence encoding SHMT2 and/or GS. The targeting endonuclease recognizes and binds the specific chromosomal sequence and introduces a double-stranded break. In some embodiments, the double-stranded break is repaired by a non-homologous end-joining (NHEJ) repair process. Because NHEJ is error prone, a deletion, insertion, and/or substitution of at least one nucleotide may occur, thereby disrupting the reading frame of the chromosomal sequence such that no protein product is produced or a non-functional protein is produced via the disruption of, for example, the enzymatically active site of the protein. In other embodiments, the targeting endonucleases can also be used to alter a chromosomal sequence via a homologous recombination reaction by co-introducing a polynucleotide having substantial sequence identity with a portion of the targeted chromosomal sequence. In such situations, the double-stranded break introduced by the targeting endonuclease is repaired by a homology-directed repair process such that the chromosomal sequence is exchanged with the polynucleotide in a manner that results in the chromosomal sequence being changed or altered (e.g., by integration of an exogenous sequence).

### (a) Targeting Endonucleases

A variety of targeting endonucleases can be used to modify the chromosomal sequences encoding SHMT2 and/or GS. The targeting endonuclease can be a naturally-occurring protein or an engineered protein. Suitable targeting endonucleases include, without limit, zinc finger nucleases (ZFNs), CRISPR nucleases, transcription activator-like effector (TALE) nucleases (TALENs), meganucleases, chimeric nucleases, site-specific endonucleases, and artificial targeted DNA double strand break inducing agents.

### (i) Zinc Finger Nucleases

In specific embodiments, the targeting endonuclease can be a pair of zinc finger nucleases (ZFNs). ZFNs bind to specific targeted sequences and introduce a double-stranded break into a targeted cleavage site. Typically, a ZFN comprises a DNA binding domain (i.e., zinc fingers) and a cleavage domain (i.e., nuclease), each of which is described below.

DNA binding domain. A DNA binding domains or the zinc fingers can be engineered to recognize and bind to any nucleic acid sequence of choice. See, for example, Beerli et al. (2002) Nat. Biotechnol. 20:135-141; Pabo et al. (2001) Ann. Rev. Biochem. 70:313-340; Isalan et al. (2001) Nat. Biotechnol. 19:656-660; Segal et al. (2001) Curr. Opin. Biotechnol. 12:632-637; Choo et al. (2000) Curr. Opin. Struct. Biol. 10:411-416; Zhang et al. (2000) J. Biol. Chem. 275(43):33850-33860; Doyon et al. (2008) Nat. Biotechnol. 26:702-708; and Santiago et al. (2008) Proc. Natl. Acad. Sci. USA 105:5809-5814. An engineered zinc finger binding domain may have a novel binding specificity compared to a naturally-occurring zinc finger protein. Engineering methods include, but are not limited to, rational design and various types of selection. Rational design includes, for example, using databases comprising doublet, triplet, and/or quadruplet nucleotide sequences and individual zinc finger amino acid sequences, in which each doublet, triplet or quadruplet nucleotide sequence is associated with one or more amino acid sequences of zinc fingers which bind the particular triplet or quadruplet sequence. See, for example, U.S. Pat. Nos. 6,453,242 and 6,534,261, the disclosures of which are incorporated by reference herein in their entireties. As an example, the algorithm described in U.S. Pat. No. 6,453,242 can be used to design a zinc finger binding domain to target a preselected sequence. Alternative methods, such as rational design using a nondegenerate recognition code table may also be used to design a zinc finger binding domain to target a specific sequence (Sera et al. (2002) Biochemistry 41:7074-7081). Publicly available web-based tools for identifying potential target sites in DNA sequences as well as designing zinc finger binding domains are known in the art. For example, tools for identifying potential target sites in DNA sequences can be found at zincfingertools.org. Tools for designing zinc finger binding domains can be found at zifit.partners.org/ZiFiT. (See also, Mandell et al. (2006) Nuc. Acid Res. 34:W516-W523; Sander et al. (2007) Nuc. Acid Res. 35:W599-W605.)

A zinc finger binding domain can be designed to recognize and bind a DNA sequence ranging from about 3 nucleotides to about 21 nucleotides in length. In one embodiment, the zinc finger binding domain can be designed to recognize and bind a DNA sequence ranging from about 9 to about 18 nucleotides in length. In general, the zinc finger binding domains of the zinc finger nucleases used herein comprise at least three zinc finger recognition regions or zinc fingers, wherein each zinc finger binds 3 nucleotides. In one embodiment, the zinc finger binding domain comprises four zinc finger recognition regions. In another embodiment, the zinc finger binding domain comprises five zinc finger recognition regions. In still another embodiment, the zinc finger binding domain comprises six zinc finger recognition regions. A zinc finger binding domain can be designed to bind to any suitable target DNA sequence. See for example, U.S. Pat. Nos. 6,607,882; 6,534,261 and 6,453,242, the disclosures of which are incorporated by reference herein in their entireties.

Exemplary methods of selecting a zinc finger recognition region include phage display and two-hybrid systems, which are described in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237, each of which is incorporated by reference herein in its entirety. In addition, enhancement of binding specificity for zinc finger binding domains has been described, for example, in WO 02/077227, the entire disclosure of which is incorporated herein by reference.

Zinc finger binding domains and methods for design and construction of fusion proteins (and polynucleotides encoding same) are known to those of skill in the art and are described in detail in, for example, U.S. Pat. No. 7,888,121, which is incorporated by reference herein in its entirety. Zinc finger recognition regions and/or multi-fingered zinc finger proteins can be linked together using suitable linker sequences, including for example, linkers of five or more amino acids in length. See, U.S. Pat. Nos. 6,479,626; 6,903,185; and 7,153,949, the disclosures of which are incorporated by reference herein in their entireties, for non-limiting examples of linker sequences of six or more amino acids in length. The zinc finger binding domain described herein may include a combination of suitable linkers between the individual zinc fingers of the protein.

Cleavage domain. A zinc finger nuclease also includes a cleavage domain. The cleavage domain portion of the zinc finger nuclease can be obtained from any endonuclease or exonuclease. Non-limiting examples of endonucleases from which a cleavage domain can be derived include, but are not limited to, restriction endonucleases and homing endonucleases. See, for example, New England Biolabs Catalog or Belfort et al. (1997) Nucleic Acids Res. 25:3379-3388. Additional enzymes that cleave DNA are known (e.g., S1 Nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) can be used as a source of cleavage domains.

A cleavage domain also can be derived from an enzyme or portion thereof, as described above, that requires dimerization for cleavage activity. Two zinc finger nucleases can be required for cleavage, as each nuclease comprises a monomer of the active enzyme dimer. Alternatively, a single zinc finger nuclease can comprise both monomers to create an active enzyme dimer. As used herein, an “active enzyme dimer” is an enzyme dimer capable of cleaving a nucleic acid molecule. The two cleavage monomers can be derived from the same endonuclease (or functional fragments thereof), or each monomer can be derived from a different endonuclease (or functional fragments thereof).

When two cleavage monomers are used to form an active enzyme dimer, the recognition sites for the two zinc fingers are preferably disposed such that binding of the two zinc fingers to their respective recognition sites places the cleavage monomers in a spatial orientation to each other that allows the cleavage monomers to form an active enzyme dimer, e.g., by dimerizing. As a result, the near edges of the recognition sites can be separated by about 5 to about 18 nucleotides. For instance, the near edges can be separated by about 5, 6, 7, 8, 9, 10, 11, 12, 13, 14, 15, 16, 17 or 18 nucleotides. It will however be understood that any integral number of nucleotides or nucleotide pairs can intervene between two recognition sites (e.g., from about 2 to about 50 nucleotide pairs or more). The near edges of the recognition sites of the zinc finger nucleases, such as for example those described in detail herein, can be separated by 6 nucleotides. In general, the site of cleavage lies between the recognition sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme FokI catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31978-31982. Thus, a zinc finger nuclease can comprise the cleavage domain from at least one Type IIS restriction enzyme and one or more zinc finger binding domains, which may or may not be engineered. Exemplary Type IIS restriction enzymes are described for example in International Publication WO 07/014,275, the disclosure of which is incorporated by reference herein in its entirety. Additional restriction enzymes also contain separable binding and cleavage domains, and these also are contemplated by the present disclosure. See, for example, Roberts et al. (2003) Nucleic Acids Res. 31:418-420.

An exemplary Type IIS restriction enzyme, whose cleavage domain is separable from the binding domain, is FokI. This particular enzyme is active as a dimer (Bitinaite et al. (1998) Proc. Natl. Acad. Sci. USA 95: 10, 570-10, 575). Accordingly, for the purposes of the present disclosure, the portion of the FokI enzyme used in a zinc finger nuclease is considered a cleavage monomer. Thus, for targeted double-stranded cleavage using a FokI cleavage domain, two zinc finger nucleases, each comprising a FokI cleavage monomer, can be used to reconstitute an active enzyme dimer. Alternatively, a single polypeptide molecule containing a zinc finger binding domain and two FokI cleavage monomers can also be used.

In certain embodiments, the cleavage domain comprises one or more engineered cleavage monomers that minimize or prevent homodimerization. By way of non-limiting example, amino acid residues at positions 446, 447, 479, 483, 484, 486, 487, 490, 491, 496, 498, 499, 500, 531, 534, 537, and 538 of FokI are all targets for influencing dimerization of the FokI cleavage half-domains. Exemplary engineered cleavage monomers of FokI that form obligate heterodimers include a pair in which a first cleavage monomer includes mutations at amino acid residue positions 490 and 538 of FokI and a second cleavage monomer that includes mutations at amino-acid residue positions 486 and 499.

Thus, in one embodiment of the engineered cleavage monomers, a mutation at amino acid position 490 replaces Glu (E) with Lys (K); a mutation at amino acid residue 538 replaces Iso (I) with Lys (K); a mutation at amino acid residue 486 replaces Gln (Q) with Glu (E); and a mutation at position 499 replaces Iso (I) with Lys (K). Specifically, the engineered cleavage monomers can be prepared by mutating positions 490 from E to K and 538 from I to K in one cleavage monomer to produce an engineered cleavage monomer designated “E490K:I538K” and by mutating positions 486 from Q to E and 499 from I to K in another cleavage monomer to produce an engineered cleavage monomer designated “Q486E:I499K.” The above described engineered cleavage monomers are obligate heterodimer mutants in which aberrant cleavage is minimized or abolished. Engineered cleavage monomers can be prepared using a suitable method, for example, by site-directed mutagenesis of wild-type cleavage monomers (FokI) as described in U.S. Pat. No. 7,888,121, which is incorporated herein in its entirety.

Additional domains. In some embodiments, the zinc finger nuclease further comprises at least one nuclear localization sequence (NLS). A NLS is an amino acid sequence which facilitates targeting the zinc finger nuclease protein into the nucleus to introduce a double stranded break at the target sequence in the chromosome. Nuclear localization signals are known in the art (see, e.g., Lange et al., J. Biol. Chem., 2007, 282:5101-5105). Non-limiting examples of nuclear localization signals include PKKKRKV (SEQ ID NO:1), PKKKRRV (SEQ ID NO:2), KRPAATKKAGQAKKKK (SEQ ID NO:3), YGRKKRRQRRR (SEQ ID NO:4), RKKRRQRRR (SEQ ID NO:5), PAAKRVKLD (SEQ ID NO:6), RQRRNELKRSP (SEQ ID NO:7), VSRKRPRP (SEQ ID NO:8), PPKKARED (SEQ ID NO:9), PQPKKKPL (SEQ ID NO:10), SALIKKKKKMAP (SEQ ID NO:11), PKQKKRK (SEQ ID NO: 12), RKLKKKIKKL (SEQ ID NO:13), REKKKFLKRR (SEQ ID NO:14), KRKGDEVDGVDEVAKKKSKK (SEQ ID NO:15), RKCLQAGMNLEARKTKK (SEQ ID NO: 16), NOSSNFGPMKGGNFGGRSSGPYGGGGQYFAKPRNQGGY (SEQ ID NO: 17), and RMRIZFKNKGKDTAELRRRRVEVSVELRKAKKDEQILKRRNV (SEQ ID NO: 18). The NLS can be located at the N-terminus, the C-terminus, or in an internal location of the zinc finger nuclease.

In additional embodiments, the zinc finger nuclease can also comprise at least one cell-penetrating domain. Examples of suitable cell-penetrating domains include, without limit, GRKKRRQRRRPPQPKKKRKV (SEQ ID NO:19), PLSSIFSRIGDPPKKKRKV (SEQ ID NO:20), GALFLGWLGAAGSTMGAPKKKRKV (SEQ ID NO:21), GALFLGFLGAAGSTMGAWSQPKKKRKV (SEQ ID NO:22), KETWWETWWTEWSQPKKKRKV (SEQ ID NO:23), YARAAARQARA (SEQ ID NO: 24), THRLPRRRRRR (SEQ ID NO:25), GGRRARRRRRR (SEQ ID NO:26), RRQRRTSKLMKR (SEQ ID NO:27), GWTLNSAGYLLGKINLKALAALAKKIL (SEQ ID NO: 28), KALAWEAKLAKALAKALAKHLAKALAKALKCEA (SEQ ID NO:29), and RQIKIWFQNRRMKWKK (SEQ ID NO:30). The cell-penetrating domain can be located at the N-terminus, the C-terminus, or in an internal location of the zinc finger nuclease.

In still other embodiments, the zinc finger nuclease can further comprise at least one marker domain. Non-limiting examples of marker domains include fluorescent proteins, purification tags, and epitope tags. In one embodiment, the marker domain can be a fluorescent protein. Non limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreen1), yellow fluorescent proteins (e.g. YFP, EYFP, Citrine, Venus, YPet, PhiYFP, ZsYellow1), blue fluorescent proteins (e.g. EBFP, EBFP2, Azurite, mKalama1, GFPuv, Sapphire, T-sapphire), cyan fluorescent proteins (e.g. ECFP, Cerulean, CyPet, AmCyan1, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In another embodiment, the marker domain can be a purification tag and/or an epitope tag. Suitable tags include, but are not limited to, poly(His) tag, FLAG (or DDK) tag, Halo tag, AcV5 tag, AU1 tag, AU5 tag, biotin carboxyl carrier protein (BCCP), calmodulin binding protein (CBP), chitin binding domain (CBD), E tag, E2 tag, ECS tag, exact tag, Glu-Glu tag, glutathione-S-transferase (GST), HA tag, HSV tag, KT3 tag, maltose binding protein (MBP), MAP tag, Myc tag, NE tag, NusA tag, PDZ tag, S tag, S1 tag, SBP tag, Softag 1 tag, Softag 3 tag, Spot tag, Strep tag, SUMO tag, T7 tag, tandem affinity purification (TAP) tag, thioredoxin (TRX), V5 tag, VSV-G tag, and Xa tag. The marker domain can be located at the N-terminus, the C-terminus, or in an internal location of the zinc finger nuclease.

The at least one nuclear localization signal, at least one cell-penetrating domain, and/or at least one marker domain can be linked directly to the zinc finger nuclease via one or more chemical bonds (e.g., covalent bonds). Alternatively, the at least one nuclear localization signal, at least one cell-penetrating domain, and/or at least one marker domain, can be linked indirectly to the zinc finger nuclease via one or more linkers. Suitable linkers include amino acids, peptides, nucleotides, nucleic acids, organic linker molecules (e.g., maleimide derivatives, N-ethoxybenzylimidazole, biphenyl-3,4′,5-tricarboxylic acid, p-aminobenzyloxycarbonyl, and the like), disulfide linkers, and polymer linkers (e.g., PEG). The linker can include one or more spacing groups including, but not limited to alkylene, alkenylene, alkynylene, alkyl, alkenyl, alkynyl, alkoxy, aryl, heteroaryl, aralkyl, aralkenyl, aralkynyl and the like. The linker can be neutral, or carry a positive or negative charge. Additionally, the linker can be cleavable such that the linker's covalent bond that connects the linker to another chemical group can be broken or cleaved under certain conditions, including pH, temperature, salt concentration, light, a catalyst, or an enzyme. In some embodiments, the linker can be a peptide linker. The peptide linker can be a flexible amino acid linker or a rigid amino acid linker. Additional examples of suitable linkers are well known in the art and programs to design linkers are readily available (Crasto et al., Protein Eng., 2000, 13 (5): 309-312).

### (ii) CRISPR Ribonucleoproteins (RNPs)

In other embodiments, the targeting endonuclease can be a Clustered Regularly Interspersed Short Palindromic Repeat (CRISPR) nuclease. CRISPR nucleases are RNA-guided nucleases derived from bacterial or archaeal CRISPR/CRISPR-associated (Cas) systems. A CRISPR RNP system comprises a CRISPR nuclease and a guide RNA.

Nuclease. The CRISPR nuclease can be derived from a type I (i.e., IA, IB, IC, ID, IE, or IF), type II (i.e., IIA, IIB, or IIC), type III (i.e., IIIA or IIIB), type V, or type VI CRISPR system, which are present in various bacteria and archaea. For example, the CRISPR nuclease can be from Streptococcus sp. (e.g., S. pyogenes, S. thermophilus, S. pasteurianus), Campylobacter sp. (e.g., Campylobacter jejuni), Francisella sp. (e.g., Francisella novicida), Acaryochloris sp., Acetohalobium sp., Acidaminococcus sp., Acidithiobacillus sp., Alicyclobacillus sp., Allochromatium sp., Ammonifex sp., Anabaena sp., Arthrospira sp., Bacillus sp., Burkholderiales sp., Caldicelulosiruptor sp., Candidatus sp., Clostridium sp., Crocosphaera sp., Cyanothece sp., Exiguobacterium sp., Finegoldia sp., Ktedonobacter sp., Lachnospiraceae sp., Lactobacillus sp., Lyngbya sp., Marinobacter sp., Methanohalobium sp., Microscilla sp., Microcoleus sp., Microcystis sp., Natranaerobius sp., Neisseria sp., Nitrosococcus sp., Nocardiopsis sp., Nodularia sp., Nostoc sp., Oscillatoria sp., Polaromonas sp., Pelotomaculum sp., Pseudoalteromonas sp., Petrotoga sp., Prevotella sp., Staphylococcus sp., Streptomyces sp., Streptosporangium sp., Synechococcus sp., Thermosipho sp., or Verrucomicrobia sp. In other embodiments, the CRISPR nuclease can be derived from an archaeal CRISPR system, a CRISPR/CasX system, or a CRISPR/CasY system (Burstein et al., Nature, 2017, 542 (7640): 237-241).

In some embodiments, the CRISPR nuclease can be derived from a type II CRISPR nuclease. For example, the type II CRISPR nuclease can be a Cas9 protein. Suitable Cas9 nucleases include Streptococcus pyogenes Cas9 (SpCas9), Francisella novicida Cas9 (FnCas9), Staphylococcus aureus (SaCas9), Streptococcus thermophilus Cas9 (StCas9), Streptococcus pasteurianus (SpaCas9), Campylobacter jejuni Cas9 (CjCas9), Neisseria meningitis Cas9 (NmCas9), or Neisseria cinerea Cas9 (NcCas9). In other embodiments, the CRISPR nuclease can be derived from a type V CRISPR nuclease, such as a Cpf1 nuclease. Suitable Cpf1 nucleases include Francisella novicida Cpf1 (FnCpf1), Acidaminococcus sp. Cpf1 (AsCpf1), or Lachnospiraceae bacterium ND2006 Cpf1 (LbCpf1). In yet another embodiment, the CRISPR nuclease can be derived from a type VI CRISPR nuclease, e.g., Leptotrichia wadei Cas13a (LwaCas13a) or Leptotrichia shahii Cas13a (LshCas13a).

The CRISPR nuclease can be a wild type CRISPR nuclease, a modified CRISPR nuclease, or a fragment of a wild type or modified CRISPR nuclease. The CRISPR nuclease can be modified to increase nucleic acid binding affinity and/or specificity, alter enzymatic activity, and/or change another property of the protein. For example, nuclease (i.e., DNase, RNase) domains of the CRISPR nuclease can be modified, deleted, or inactivated. The CRISPR nuclease can be truncated to remove domains that are not essential for the function of the nuclease.

CRISPR nucleases comprise two nuclease domains. For example, a Cas9 nuclease comprises a HNH domain, which cleaves the guide RNA complementary strand, and a RuvC domain, which cleaves the non-complementary strand; a Cpf1 nuclease comprises a RuvC domain and a NUC domain; and a Cas13a nuclease comprises two HNEPN domains. When both nuclease domains are functional, CRISPR nuclease introduces a double-stranded break. Either nuclease domain can be inactivated by one or more mutations and/or deletions, thereby creating a variant that introduces a single-strand break in one strand of the double-stranded sequence. For example, one or more mutations in the RuvC domain of Cas9 nuclease (e.g., D10A, D8A, E762A, and/or D986A) results in an HNH nickase that nicks the guide RNA complementary strand; and one or more mutations in the HNH domain of Cas9 nuclease (e.g., H840A, H559A, N854A, N856A, and/or N863A) results in a RuvC nickase that nicks the guide RNA non-complementary strand. Comparable mutations can convert Cpf1 and Cas13a nucleases to nickases. Two CRISPR nickases targeted to opposites strands of a chromosomal sequence (via a pair of offset guide RNAs) can be used in combination to create a double-stranded break in the chromosomal sequence. Dual CRISPR nickase RNPs can increase target specificity and reduce off target effects.

Additional domains. The CRISPR nuclease can further comprise at least one nuclear localization sequence (NLS). A NLS is an amino acid sequence which facilitates targeting the zinc finger nuclease protein into the nucleus to introduce a double stranded break at the target sequence in the chromosome. Nuclear localization signals are known in the art (see, e.g., Lange et al., J. Biol. Chem., 2007, 282:5101-5105). Non-limiting examples of nuclear localization signals include PKKKRKV (SEQ ID NO:1), PKKKRRV (SEQ ID NO:2), KRPAATKKAGQAKKKK (SEQ ID NO:3), YGRKKRRQRRR (SEQ ID NO:4), RKKRRQRRR (SEQ ID NO:5), PAAKRVKLD (SEQ ID NO:6), RQRRNELKRSP (SEQ ID NO:7), VSRKRPRP (SEQ ID NO:8), PPKKARED (SEQ ID NO:9), PQPKKKPL (SEQ ID NO:10), SALIKKKKKMAP (SEQ ID NO:11), PKQKKRK (SEQ ID NO: 12), RKLKKKIKKL (SEQ ID NO:13), REKKKFLKRR (SEQ ID NO:14), KRKGDEVDGVDEVAKKKSKK (SEQ ID NO:15), RKCLQAGMNLEARKTKK (SEQ ID NO: 16), NOSSNFGPMKGGNFGGRSSGPYGGGGQYFAKPRNQGGY (SEQ ID NO: 17), and RMRIZFKNKGKDTAELRRRRVEVSVELRKAKKDEQILKRRNV (SEQ ID NO: 18). The NLS can be located at the N-terminus, the C-terminus, or in an internal location of the CRISPR nuclease.

In additional embodiments, the CRISPR nuclease can also comprise at least one cell-penetrating domain. Examples of suitable cell-penetrating domains include, without limit, GRKKRRQRRRPPQPKKKRKV (SEQ ID NO:19), PLSSIFSRIGDPPKKKRKV (SEQ ID NO:20), GALFLGWLGAAGSTMGAPKKKRKV (SEQ ID NO:21), GALFLGFLGAAGSTMGAWSQPKKKRKV (SEQ ID NO:22), KETWWETWWTEWSQPKKKRKV (SEQ ID NO:23), YARAAARQARA (SEQ ID NO: 24), THRLPRRRRRR (SEQ ID NO:25), GGRRARRRRRR (SEQ ID NO:26), RRQRRTSKLMKR (SEQ ID NO:27), GWTLNSAGYLLGKINLKALAALAKKIL (SEQ ID NO: 28), KALAWEAKLAKALAKALAKHLAKALAKALKCEA (SEQ ID NO:29), and RQIKIWFQNRRMKWKK (SEQ ID NO:30). The cell-penetrating domain can be located at the N-terminus, the C-terminus, or in an internal location of the CRISPR protein.

In still other embodiments, the CRISPR nuclease can further comprise at least one marker domain. Non-limiting examples of marker domains include fluorescent proteins, purification tags, and epitope tags. In one embodiment, the marker domain can be a fluorescent protein. Non limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreen1), yellow fluorescent proteins (e.g. YFP, EYFP, Citrine, Venus, YPet, PhiYFP, ZsYellow1), blue fluorescent proteins (e.g. EBFP, EBFP2, Azurite, mKalama1, GFPuv, Sapphire, T-sapphire), cyan fluorescent proteins (e.g. ECFP, Cerulean, CyPet, AmCyan1, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In another embodiment, the marker domain can be a purification tag and/or an epitope tag. Suitable tags include, but are not limited to, poly(His) tag, FLAG (or DDK) tag, Halo tag, AcV5 tag, AU1 tag, AU5 tag, biotin carboxyl carrier protein (BCCP), calmodulin binding protein (CBP), chitin binding domain (CBD), E tag, E2 tag, ECS tag, exact tag, Glu-Glu tag, glutathione-S-transferase (GST), HA tag, HSV tag, KT3 tag, maltose binding protein (MBP), MAP tag, Myc tag, NE tag, NusA tag, PDZ tag, S tag, S1 tag, SBP tag, Softag 1 tag, Softag 3 tag, Spot tag, Strep tag, SUMO tag, T7 tag, tandem affinity purification (TAP) tag, thioredoxin (TRX), V5 tag, VSV-G tag, and Xa tag. The marker domain can be located at the N-terminus, the C-terminus, or in an internal location of the CRISPR nuclease.

The at least one nuclear localization signal, at least one cell-penetrating domain, and/or at least one marker domain can be linked directly to the CRISPR nuclease via one or more chemical bonds (e.g., covalent bonds). Alternatively, the at least one nuclear localization signal, at least one cell-penetrating domain, and/or at least one marker domain, can be linked indirectly to the CRISPR nuclease via one or more linkers. Suitable linkers include amino acids, peptides, nucleotides, nucleic acids, organic linker molecules (e.g., maleimide derivatives, N-ethoxybenzylimidazole, biphenyl-3,4′,5-tricarboxylic acid, p-aminobenzyloxycarbonyl, and the like), disulfide linkers, and polymer linkers (e.g., PEG). The linker can include one or more spacing groups including, but not limited to alkylene, alkenylene, alkynylene, alkyl, alkenyl, alkynyl, alkoxy, aryl, heteroaryl, aralkyl, aralkenyl, aralkynyl and the like. The linker can be neutral, or carry a positive or negative charge. Additionally, the linker can be cleavable such that the linker's covalent bond that connects the linker to another chemical group can be broken or cleaved under certain conditions, including pH, temperature, salt concentration, light, a catalyst, or an enzyme. In some embodiments, the linker can be a peptide linker. The peptide linker can be a flexible amino acid linker or a rigid amino acid linker. Additional examples of suitable linkers are well known in the art and programs to design linkers are readily in the art.

Guide RNA. A CRISPR nuclease is guided to its target site by a guide RNA. The guide RNA hybridizes with the target site and interacts with the CRISPR nuclease to direct the CRISPR nuclease to the target site in the chromosomal sequence. The target site has no sequence limitation except that the sequence is bordered by a protospacer adjacent motif (PAM). CRISPR proteins from different bacterial species recognize different PAM sequences. For example, PAM sequences include 5′-NGG (SpCas9, FnCAs9), 5′-NGRRT (SaCas9), 5′-NNAGAAW (StCas9), 5′-NNNNGATT (NmCas9), 5-NNNNRYAC (CjCas9), and 5′-TTTV (Cpf1), wherein N is defined as any nucleotide, R is defined as either G or A, W is defined as either A or T, Y is defined an either C or T, and Vis defined as A, C, or G. Cas9 PAMs are located 3′ of the target site, and cpf1 PAMs are located 5′ of the target site.

A guide RNA comprises three regions: a first region at the 5′ end that is complementary to sequence at the target site, a second internal region that forms a stem loop structure, and a third 3′ region that remains essentially single-stranded. The first region of each guide RNA is different such that each guide RNA guides a CRISPR nuclease to a specific target site. The second and third regions (also called the scaffold region) of each guide RNA can be the same in all guide RNAs.

The first region of the guide RNA is complementary to sequence (i.e., protospacer sequence) at the target site such that the first region of the guide RNA can base pair with sequence at the target site. The complementarity between the first region (i.e., crRNA) of the guide RNA and the target sequence can be at least 80%, at least 85%, at least 90%, at least 95%, or more. In general, there are no mismatches between the sequence of the first region of the guide RNA and the sequence at the target site (i.e., the complementarity is total). In various embodiments, the first region of the guide RNA can comprise from about 10 nucleotides to more than about 25 nucleotides. For example, the region of base pairing between the first region of the guide RNA and the target site in the chromosomal sequence can be about 10, 11, 12, 13, 14, 15, 16, 17, 18, 19, 20, 22, 23, 24, 25, or more than 25 nucleotides in length. In exemplary embodiments, the first region of the guide RNA is about 19, 20, or 21 nucleotides in length.

The guide RNA also comprises a second region that forms a secondary structure. In some embodiments, the secondary structure comprises a stem (or hairpin) and a loop. The length of the loop and the stem can vary. For example, the loop can range from about 3 to about 10 nucleotides in length, and the stem can range from about 6 to about 20 base pairs in length. The stem can comprise one or more bulges of 1 to about 10 nucleotides. Thus, the overall length of the second region can range from about 16 to about 60 nucleotides in length. In an exemplary embodiment, the loop is about 4 nucleotides in length and the stem comprises about 12 base pairs.

The guide RNA also comprises a third region at the 3′ end that remains essentially single-stranded. Thus, the third region has no complementarity to any chromosomal sequence in the cell of interest and has no complementarity to the rest of the guide RNA. The length of the third region can vary. In general, the third region is more than about 4 nucleotides in length. For example, the length of the third region can range from about 5 to about 60 nucleotides in length.

The combined length of the second and third regions (or scaffold) of the guide RNA can range from about 30 to about 120 nucleotides in length. In one aspect, the combined length of the second and third regions of the guide RNA range from about 70 to about 100 nucleotides in length.

In some embodiments, the guide RNA comprises one molecule comprising all three regions. In other embodiments, the guide RNA can comprise two separate molecules. The first RNA molecule can comprise the first (5′) region of the guide RNA and one half of the “stem” of the second region of the guide RNA. The second RNA molecule can comprise the other half of the “stem” of the second region of the guide RNA and the third region of the guide RNA. Thus, in this embodiment, the first and second RNA molecules each contain a sequence of nucleotides that are complementary to one another. For example, in one embodiment, the first and second RNA molecules each comprise a sequence (of about 6 to about 20 nucleotides) that base pairs to the other sequence to form a functional guide RNA.

(iii) Other Targeting Endonucleases

In further embodiments, the targeting endonuclease can be a meganuclease. Meganucleases are endodeoxyribonucleases characterized by long recognition sequences, i.e., the recognition sequence generally ranges from about 12 base pairs to about 40 base pairs. As a consequence of this requirement, the recognition sequence generally occurs only once in any given genome. Among meganucleases, the family of homing endonucleases named LAGLIDADG has become a valuable tool for the study of genomes and genome engineering (see, e.g., Arnould et al., 2011, Protein Eng Des Sel, 24(1-2):27-31). Other suitable meganucleases include I-CreI and I-DmoI. A meganuclease can be targeted to a specific chromosomal sequence by modifying its recognition sequence using techniques well known to those skilled in the art.

In additional embodiments, the targeting endonuclease can be a transcription activator-like effector (TALE) nuclease. TALEs are transcription factors from the plant pathogen Xanthomonas that can be readily engineered to bind new DNA targets. TALEs or truncated versions thereof may be linked to the catalytic domain of endonucleases such as FokI to create targeting endonuclease called TALE nucleases or TALENs (Sanjana et al., 2012, Nat Protoc, 7(1):171-192) and Arnould et al., 2011, Protein Engineering, Design & Selection, 24(1-2):27-31).

In alternate embodiments, the targeting endonuclease can be chimeric nuclease. Non-limiting examples of chimeric nucleases include ZF-meganucleases, TAL-meganucleases, Cas9-FokI fusions, ZF-Cas9 fusions, TAL-Cas9 fusions, and the like. Persons skilled in the art are familiar with means for generating such chimeric nuclease fusions.

In still other embodiments, the targeting endonuclease can be a site-specific endonuclease. In particular, the site-specific endonuclease can be a “rare-cutter” endonuclease whose recognition sequence occurs rarely in a genome. Alternatively, the site-specific endonuclease can be engineered to cleave a site of interest (Friedhoff et al., 2007, Methods Mol Biol 352:1110123). Generally, the recognition sequence of the site-specific endonuclease occurs only once in a genome. In alternate further embodiments, the targeting endonuclease can be an artificial targeted DNA double strand break inducing agent.

### (b) Delivery of the Targeting Endonuclease to the Cell

The method comprises introducing the targeting endonuclease into the parental cell line of interest. The targeting endonuclease can be introduced into the cells as a purified isolated protein or as a nucleic acid encoding the targeting endonuclease. The nucleic acid can be DNA or RNA. In embodiments in which the encoding nucleic acid is mRNA, the mRNA may be 5′ capped and/or 3′ polyadenylated. In embodiments in which the encoding nucleic acid is DNA, the DNA can be linear or circular. The nucleic acid can be part of a plasmid or viral vector, wherein the encoding DNA can be operably linked to a suitable promoter. Those skilled in the art are familiar with appropriate vectors, promoters, other control elements, and means of introducing the vector into the cell of interest. In embodiments in which targeting endonuclease is a CRISPR nuclease, the CRISPR nuclease system can be introduced into the cell as a gRNA-protein complex.

The targeting endonuclease molecule(s) can be introduced into the cell by a variety of means. Suitable delivery means include microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In a specific embodiment, the targeting endonuclease molecule(s) are introduced into the cell by nucleofection.

Optional Donor Polynucleotide. The method for targeted genome modification or engineering can further comprise introducing into the cell at least one donor polynucleotide comprising sequence having at least one nucleotide change relative to the target chromosomal sequence. The donor polynucleotide has substantial sequence identity to sequence at or near the targeted site in the chromosomal sequence such that the double-stranded break introduced by the targeting endonuclease can be repaired by a homology-directed repair process and the sequence of the donor polynucleotide can be inserted into or exchanged with the chromosomal sequence, thereby modifying the chromosomal sequence. For example, the donor polynucleotide can comprise a first sequence having substantial sequence identity to sequence on one side of the target site and a second sequence having substantial sequence identity to sequence on the other side of the target site. The donor polynucleotide can further comprise a donor sequence for integration into the targeted chromosomal sequence. For example, the donor sequence can be an exogenous sequence (e.g., a marker sequence) such that integration of the exogenous sequence disrupts the reading frame and inactivates the targeted chromosomal sequence.

The lengths of the first and second sequences in the donor polynucleotide that have substantial sequence identity to sequences at or near the target site in the chromosomal sequence can and will vary. In general, each of the first and second sequences in the donor polynucleotide is at least about 10 nucleotides in length. In various embodiments, the donor polynucleotide sequences having substantial sequence identity with chromosomal sequences can be about 15 nucleotides, about 20 nucleotides, about 25 nucleotides, about 30 nucleotides, about 40 nucleotides, about 50 nucleotides, about 100 nucleotides, or more than 100 nucleotides in length.

The phrase “substantial sequence identity” means that the sequences in the polynucleotide have at least about 75% sequence identity with the chromosomal sequences of interest. In some embodiments, the sequences in the polynucleotide about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with the chromosomal sequences of interest.

The length of the donor polynucleotide can and will vary. For example, the donor polynucleotide can range from about 20 nucleotides in length up to about 200,000 nucleotides in length. In various embodiments, the donor polynucleotide can range from about 20 nucleotides to about 100 nucleotides in length, from about 100 nucleotides to about 1000 nucleotides in length, from about 1000 nucleotides to about 10,000 nucleotides in length, from about 10,000 nucleotides to about 100,000 nucleotides in length, or from about 100,000 nucleotides to about 200,000 nucleotides in length.

Typically, the donor polynucleotide is DNA. The DNA can be single-stranded or double-stranded. The DNA can be linear or circular. In some embodiments, the donor polynucleotide can be an single-stranded, linear oligonucleotide comprising less than about 200 nucleotides. In other embodiments, the donor polynucleotide can be part of a vector. Suitable vectors include DNA plasmids, viral vectors, bacterial artificial chromosomes (BAC), and yeast artificial chromosomes (YAC). In still other embodiments, the donor polynucleotide can be a PCR fragment or a nucleic acid complexed with a delivery vehicle such as a liposome or poloxamer.

The donor polynucleotide(s) can be introduced into the cells at the same time as the targeting endonuclease molecule(s). Alternatively, the donor polynucleotide(s) and the targeting endonuclease molecule(s) can be introduced into the cells sequentially. The ratio of the targeting endonuclease molecule(s) to the donor polynucleotide(s) can and will vary. In general, the ratio of targeting endonuclease molecule(s) to donor polynucleotide(s) ranges from about 1:10 to about 10:1. In various embodiments, the ratio of the targeting endonuclease molecule(s) to polynucleotide(s) can be about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In one embodiment, the ratio is about 1:1.

### (c) Culturing the Cell

The method further comprises maintaining the cell under appropriate conditions such that the double-stranded break introduced by the targeting endonuclease can be repaired by (i) a non-homologous end-joining repair process such that the chromosomal sequence is modified by a deletion, insertion and/or substitution of at least one nucleotide or, optionally, (ii) a homology-directed repair process such that the chromosomal sequence is exchanged with the sequence of the polynucleotide such that the chromosomal sequence is modified. In embodiments in which nucleic acid(s) encoding the targeting endonuclease(s) is introduced into the cell, the method comprises maintaining the cell under appropriate conditions such that the cell expresses the targeting endonuclease(s).

In general, the cell is maintained under conditions appropriate for cell growth and/or maintenance. Suitable cell culture conditions are well known in the art and are described, for example, in Santiago et al. (2008) PNAS 105:5809-5814; Moehle et al. (2007) PNAS 104:3055-3060; Urnov et al. (2005) Nature 435:646-651; and Lombardo et al (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

During this step of the process, the targeting endonuclease(s) recognizes, binds, and creates a double-stranded break(s) at the targeted cleavage site(s) in the chromosomal sequence, and during repair of the double-stranded break(s) a deletion, insertion, and/or substitution of at least one nucleotide is introduced into the targeted chromosomal sequence. In specific embodiments, the targeted chromosomal sequence is inactivated.

Upon confirmation that the chromosomal sequence of interest has been modified, single cell clones can be isolated and genotyped (via DNA sequencing and/or protein analyses). Cells comprising one modified chromosomal sequence can undergo one or more additional rounds of targeted genome modification to modify additional chromosomal sequences, thereby creating double knock-out, triple knock-outs, and the like.

### (IV) Producing Recombinant Proteins

Another aspect of the present disclosure encompasses methods for producing recombinant proteins in a biologic production system. Suitable recombinant proteins are described in section (I) (c). The methods comprise expressing the recombinant protein of interest in any of the engineered cell lines described above in section (I) and purifying the expressed recombinant protein. Means for producing or manufacturing recombinant proteins are well known in the field (see, e.g., “Biopharmaceutical Production Technology”, Subramanian (ed), 2012, Wiley-VCH; ISBN: 978-3-527-33029-4).

The recombinant protein can be purified via a process comprising a step of clarification, e.g., filtration, and one or more steps of chromatography, e.g., affinity chromatography, protein A (or G) chromatography, ion exchange (i.e., cation and/or anion) chromatography.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

As used herein, the term “endogenous sequence” refers to a chromosomal sequence that is native to the cell.

The term “exogenous sequence” refers to a chromosomal sequence that is not native to the cell, or a chromosomal sequence that is moved to a different chromosomal location.

An “engineered” or “genetically modified” cell refers to a cell in which the genome has been modified or engineered, i.e., the cell contains at least one chromosomal sequence that has been engineered to contain an insertion of at least one nucleotide, a deletion of at least one nucleotide, and/or a substitution of at least one nucleotide.

The terms “genome modification” and “genome editing” refer to processes by which a specific endogenous chromosomal sequence is changed such that the chromosomal sequence is modified. The chromosomal sequence may be modified to comprise an insertion of at least one nucleotide, a deletion of at least one nucleotide, and/or a substitution of at least one nucleotide. The modified chromosomal sequence is inactivated such that no product is made. Alternatively, the chromosomal sequence can be modified such that an altered product is made.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The term “heterologous” refers to an entity that is not native to the cell or species of interest.

The terms “nucleic acid” and “polynucleotide” refer to a deoxyribonucleotide or ribonucleotide polymer, in linear or circular conformation. For the purposes of the present disclosure, these terms are not to be construed as limiting with respect to the length of a polymer. The terms can encompass known analogs of natural nucleotides, as well as nucleotides that are modified in the base, sugar and/or phosphate moieties. In general, an analog of a particular nucleotide has the same base-pairing specificity; i.e., an analog of A will base-pair with T. The nucleotides of a nucleic acid or polynucleotide may be linked by phosphodiester, phosphothioate, phosphoramidite, phosphorodiamidate bonds, or combinations thereof.

The term “nucleotide” refers to deoxyribonucleotides or ribonucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

As used herein, the terms “target site” or “target sequence” refer to a nucleic acid sequence that defines a portion of a chromosomal sequence to be modified or edited and to which a targeting endonuclease is engineered to recognize and bind, provided sufficient conditions for binding exist.

The terms “upstream” and “downstream” refer to locations in a nucleic acid sequence relative to a fixed position. Upstream refers to the region that is 5′ (i.e., near the 5′ end of the strand) to the position and downstream refers to the region that is 3′ (i.e., near the 3′ end of the strand) to the position.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14 (6): 6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

As various changes could be made in the above-described cells and methods without departing from the scope of the invention, it is intended that all matter contained in the above description and in the examples given below, shall be interpreted as illustrative and not in a limiting sense.

### EXAMPLES

The following examples illustrate certain aspects of the invention.

### Example 1: Design of Glycine-Formate Mediated Selection System

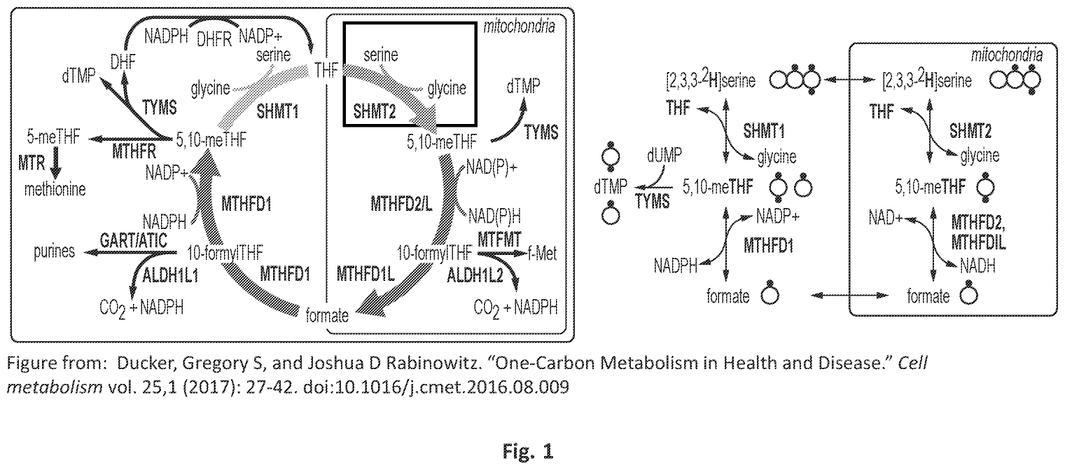

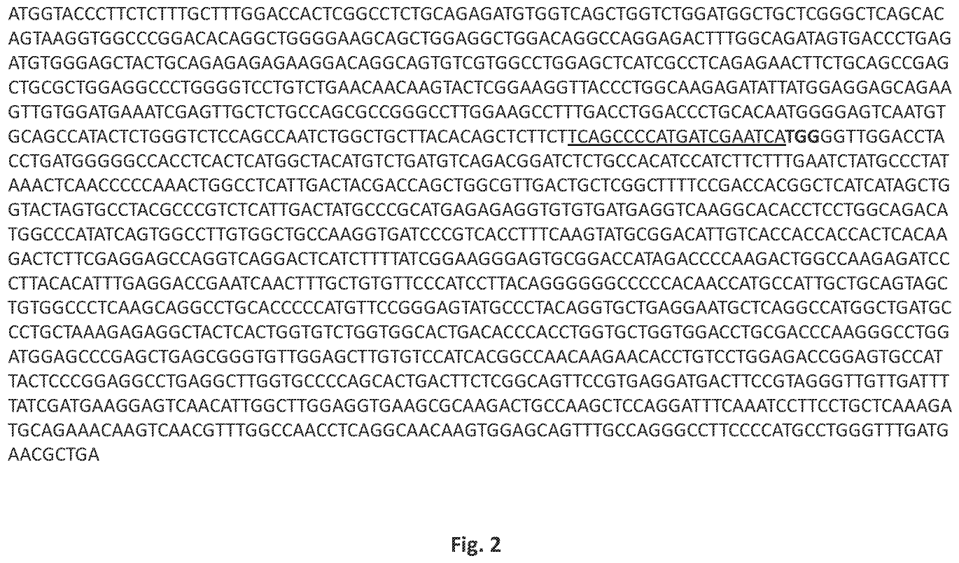

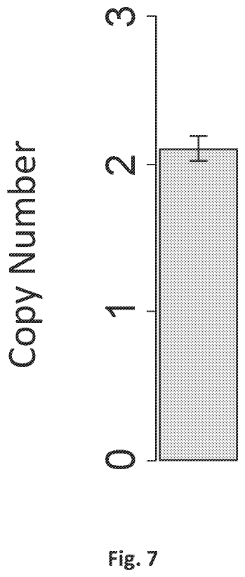

To develop a glycine-formate mediated metabolic selection system, a CHO cell line that is auxotrophic for the non-essential amino acid Glycine (Gly), was first developed. A comprehensive search to identify all genes associated with the glycine-formate synthesis pathway was conducted against the Reactome and KEGG database. The endogenous serine hydroxymethyl transferase 2 gene (SHMT2) was identified as the lone non-redundant gene responsible for Gly synthesis (FIG. 1). To generate a Gly auxotrophic CHO cell line, the Glutamine (Gln) auxotrophic, CHOZN® GS^{-/-} cell line (CHOZN®) from MilliporeSigma was utilized. The endogenous SHMT2 coding sequence (FIG. 2) was elucidated via whole genome sequencing (WGS) of the CHOZN® cell line, and it was found to be present in two copies in the CHOZN® genome, as determined by digital droplet PCR (ddPCR) analysis (FIG. 7). CRISPR/Cas9 gene editing reagents were designed to disrupt the SHMT2 gene. The CRISPR/Cas9 target sequence is underlined in FIG. 2. CHOZN® cells were cultured in EX-CELL® CD CHO Fusion medium (MilliporeSigma 14365C) supplemented with 6 mM L-Glutamine (MilliporeSigma G7513) (Fusion+Gln) under shaking conditions at 37° C. with 5% CO2. Cells were split to 0.3e6 3 days prior to the transfection. Cas9 RNPs were complexed at room temperature for 15 min by mixing 50 pmols of Cas9 (Sigma CAS9PROT-250UG) with 150 pmol of sgRNA (Sigma). 4e5 cells were transfected with a total of 200 pmol of complexed RNP using Lonza's 4DX nucleofector system with program DT-133 and SF nucleofector solution. Transfected cells were transferred into a 6-well culture flask containing 2 mL of pre-warmed EX-CELL® CD CHO Fusion medium (MilliporeSigma 14365C) supplemented with 6 mM L-Glutamine (MilliporeSigma G7513). The cells were incubated at 37° C./5% CO2 in a static environment. 48 hours post transfection, 20% of the pool (400 μL) was harvested for genomic DNA extraction (gDNA) and the gDNA was extracted using QuickExtract (Lucigen QE09050). 2.5 uL of gDNA was amplified for next-generation sequencing (NGS) using an illumina Miseq. NGS confirmed >90% of the pool was edited.

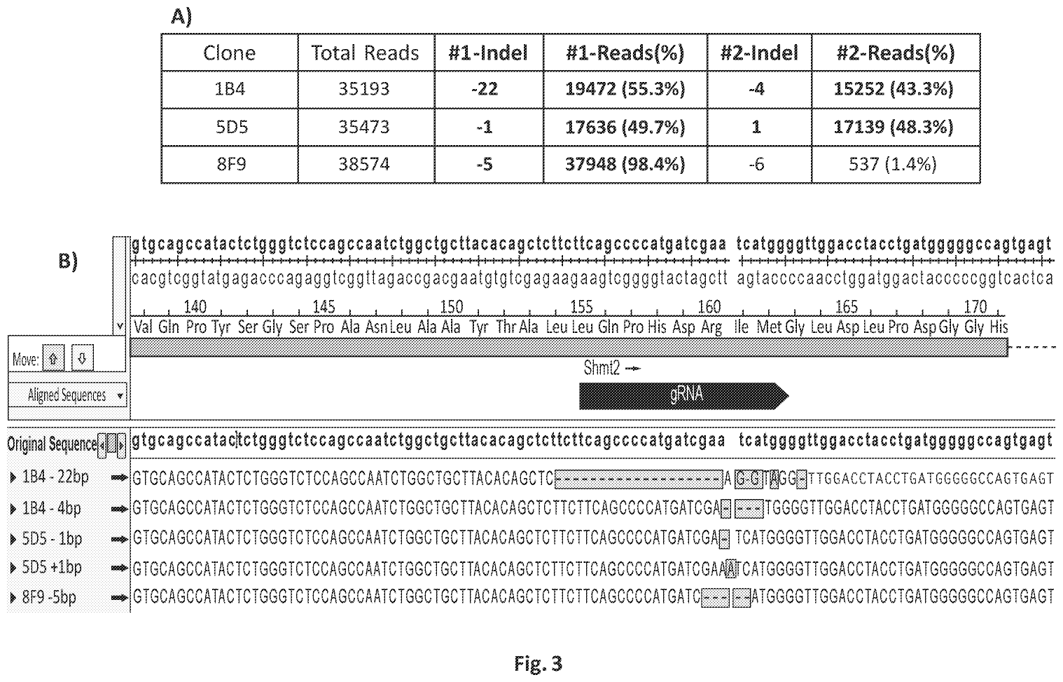

Single cell clones from the Cas9 modified pool were isolated via a florescence activated cell sorter (FACS) into 96-well culture plates. Single cell clones were evaluated via NGS to identify clones containing the successful genetic disruption of both copies of SHMT2 (FIG. 3).

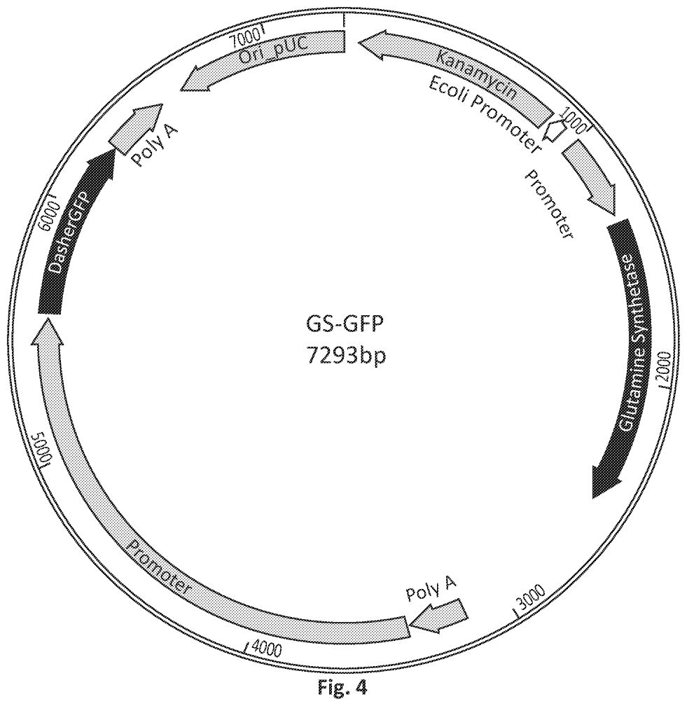

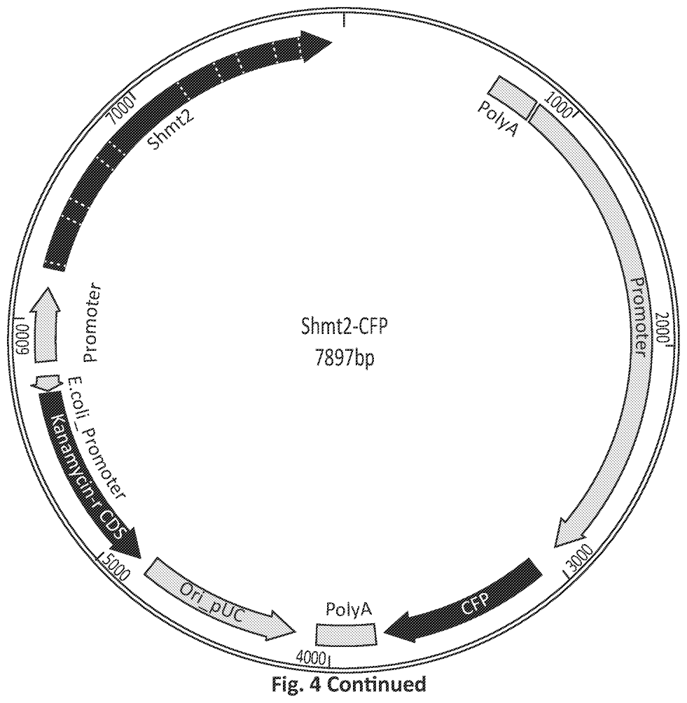

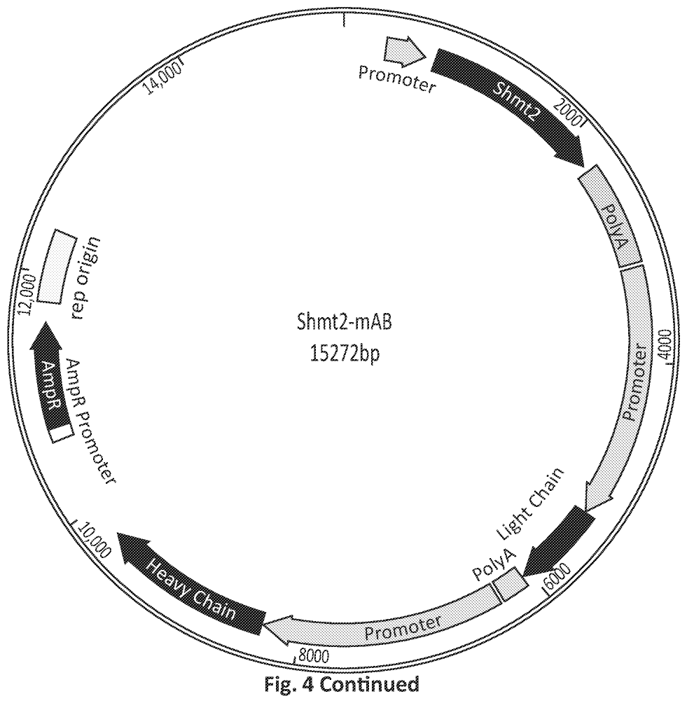

To demonstrate the efficacy of a glycine-formate mediated selection mechanism as both a stand-alone system and as one part of a dual metabolic selection system, stable selected cell populations were generated, expressing a multitude of molecules. The molecules used in the validation of this system include Cyan Fluorescent Protein (CFP), Dasher Green Fluorescent Protein (GFP), and a human IgG1CHOZN® GS^{-/-} SHMT2^{-/-} cells were cultured in Fusion+Gln+Gly+Formate (For) medium. The expression vectors used in the current work contain either a Serine Hydroxymethyltransferase 2 (SHMT2) or Glutamine Synthetase (GS) selection marker as dictated by the experimental design. Expression of murine SHMT2 (Protein serine hydroxymethyltransferase 2; Gene: SHMT2; UniProtKB ID: Q9CZN7) or murine GS (Protein: glutamine synthetase {glutamate-ammonia ligase}: Gene: GluI; UniProtKB ID: P15105) was driven by a 5′ SV40 promoter with a SV40 polyadenylation sequence at the 3′ end of the gene (FIG. 4).

CHOZN® GS^{-/-} SHMT2^{-/-} cells were cultured in Fusion +Gln +Gly +For under shaking conditions at 37° C. with 5% CO2. Cells were split to 0.3e6 3 days prior to the transfection. 1.0e6 cells per condition were transfected with 1 μg of plasmid DNA using Lonza's 4DX nucleofector system with program DT-133 and SF nucleofector solution. Transfected cells were transferred into a 6-well culture flask containing 3 mL of pre-warmed EX-CELL® CD CHO Fusion medium (MilliporeSigma 14365C) supplemented with 6 mM L-Glutamine (MilliporeSigma G7513) and 200 uM of sodium formate (Sigma 456020-25G). The cells were incubated at 37° C./5% CO2 in a static environment. 3 days post transfection, each sample was expanded to T-25's and 2 mls of media was added. On day 7, the cells reached ˜90% viability and were transferred to 15 ml conicals and spun down at 1000 rpm (˜300×g) for 5 min. The supernatant was aspirated and the cells were then washed with 5mLs of PBS and spun again for 5 min. The supernatant was aspirated and the cells were resuspended in 10-15mls of their corresponding selection media and transferred to a T-75 flask. Glutamine-based selection was conducted using Fusion −Gln, Glycine Formate-based selection was conducted using Fusion −Gly −For, and Glutamine/Glycine Formate dual selection was conducted using Fusion without Gln, Gly and For. Cell viability and viable cell density of the various selection cultures were monitored over time.

Gly deficient custom formulations of the EX-CELL® Advanced CHO Fed-batch medium (MilliporeSigma 14366C) and EX-CELL® Advanced CHO Feed (MilliporeSigma 24367C were developed (Advanced-Gly and Feed-Gly, respectively). Cellvento® 4 Feed (MilliporeSigma 1.03796.0005) was used unmodified. Stable selected cultures transfected with an IgG1 expression vector were pelleted, with selection medium being aspirated off, followed by resuspension at 3e5 viable cells/mL in Advanced-Gly for productivity analysis in fed-batch conditions. Viable cell densities and viabilities for each culture were collected every other day beginning on day 3 post-seeding. Beginning on day 3 post-seeding, 1.5 mL of a 50/50 blend of Advanced Feed-Gly and 4 Feed were added to each culture. Glucose readings were taken from each culture every other day beginning on day 5 with D-+-Glucose (MilliporeSigma G8769) being added to maintain an appropriate glucose level. Productivity was monitored over time, with fed batch titers being recorded every other day, beginning on day 8, until the cultures dropped below 70% viability. Titers were determined using interferometry on a ForteBio Octet, followed by confirmation via HPLC protein A affinity chromatography.

### Example 2

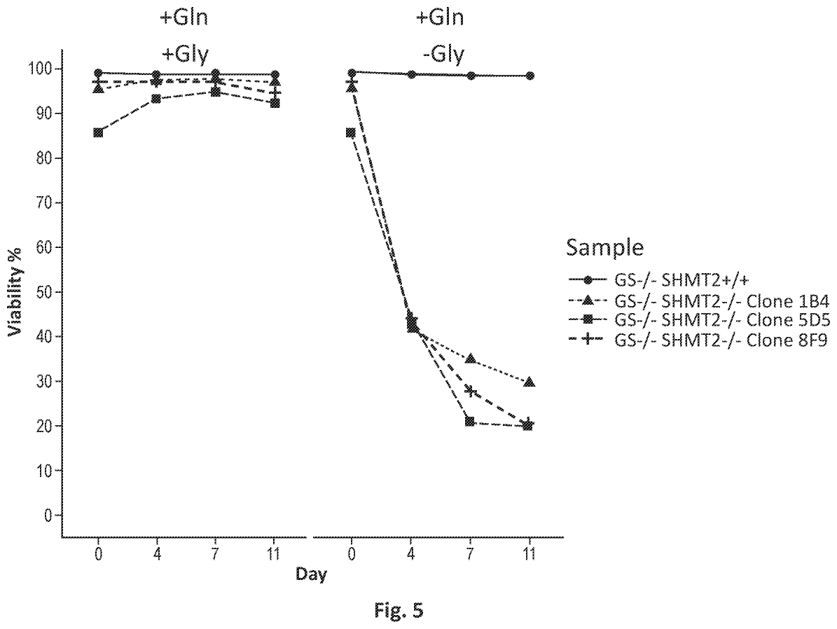

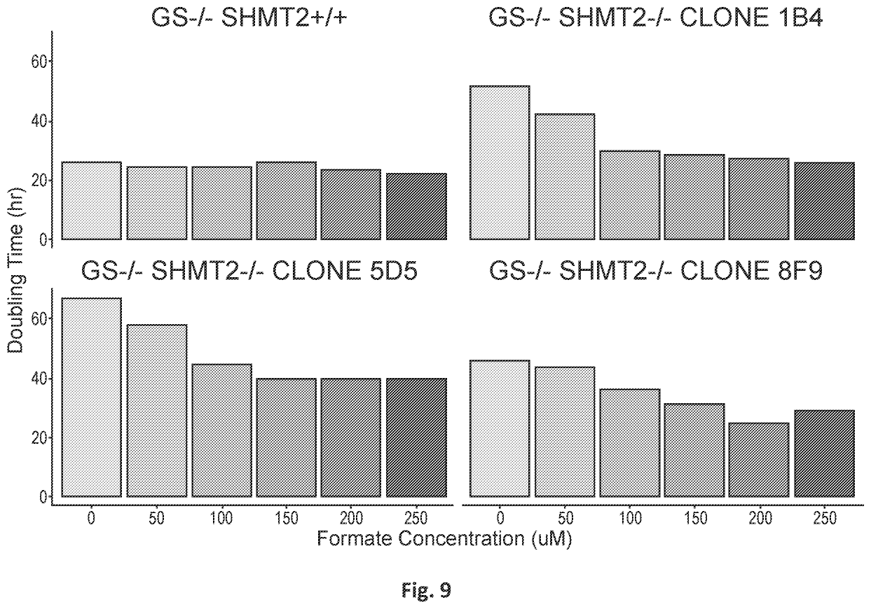

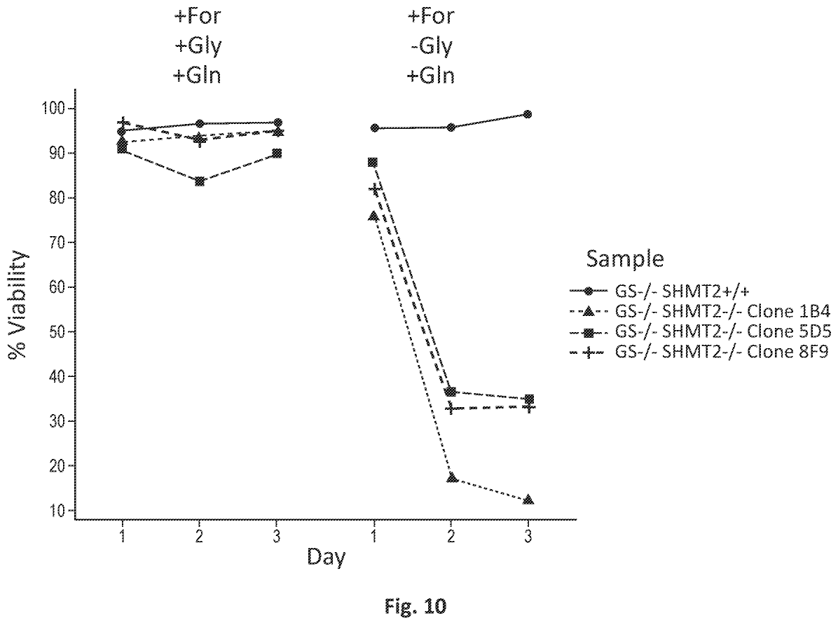

A custom formulation of the basal EX-CELL® CD CHO Fusion medium without Glycine (Gly) was developed (Fusion −Gln-Gly). CHOZN® GS^{-/-} SHMT2^{-/-} clones were cultured in Fusion +Gln +Gly +For, or Fusion+Gln −Gly −For for at least ten days. Viabilities and viable cell density measurements were taken twice per week. FIG. 5 indicates that in the absence of glycine and formate the CHOZN® GS^{-/-} SHMT2^{-/-} cells are unable to grow, however when glycine is supplemented into the media CHOZN® GS^{-/-} SHMT2^{-/-} cell growth is rescued. Importantly, the rate of SHMT2^{-/-} cell growth is significantly slower than the SHMT2^{+/+} cell line, although this can be rescued via the addition of sodium formate into the media (FIG. 9). Addition of only Formate (Fusion −Gly +For) is not sufficient for cell survival (FIG. 10).

### Example 3

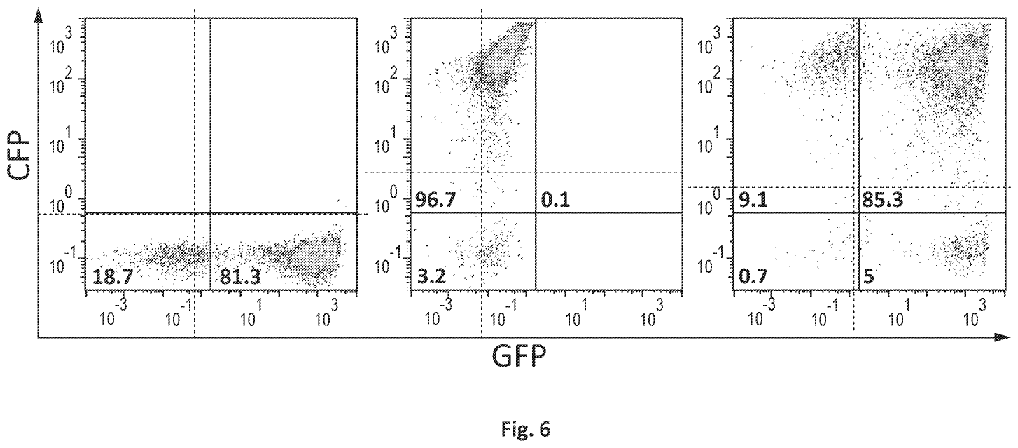

To demonstrate that stable selected cell populations were able to produce a protein of interest using the Gly/For-mediated selection system described in Example 1, cells were transfected and passaged under selective pressure in Fusion −Gly −For. A SHMT2/CFP vector was transfected into CHOZN® GS^{-/-} SHMT2^{-/-} cells. Cell growth and viability was monitored throughout selection. Cells transfected with the SHMT2/CFP plasmid and grown in Fusion +Gln −Gly −For (Gly selective media) recovered from the selective pressure. Surviving cell populations were analyzed by FACS and the mean fluorescence intensity (MFI) and percentage of CFP+ cells was measured. Cells grown in Fusion +Gln +Gly +For showed a very small percentage of CFP positive cells and a low MFI compared to the cells subjected to Gly/For-selection in Fusion +Gln −Gly −For. Results are summarized in FIGS. 6 & 8.

### Example 4

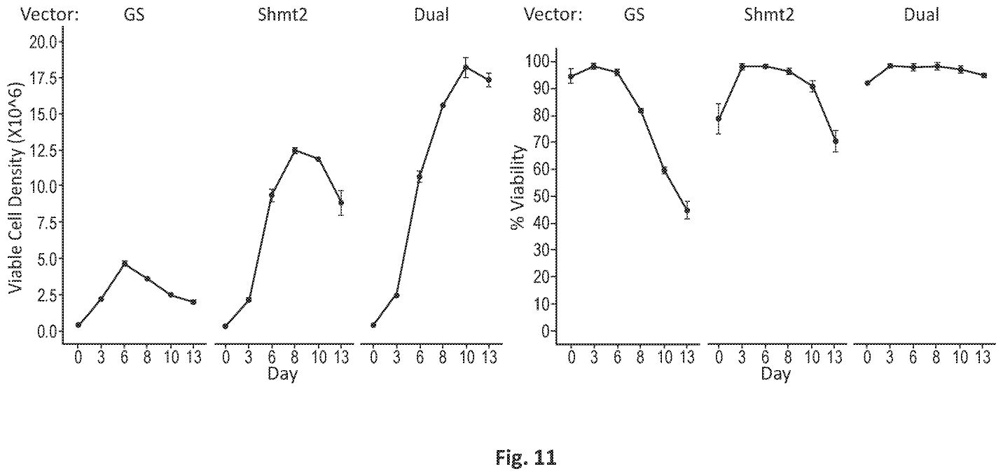

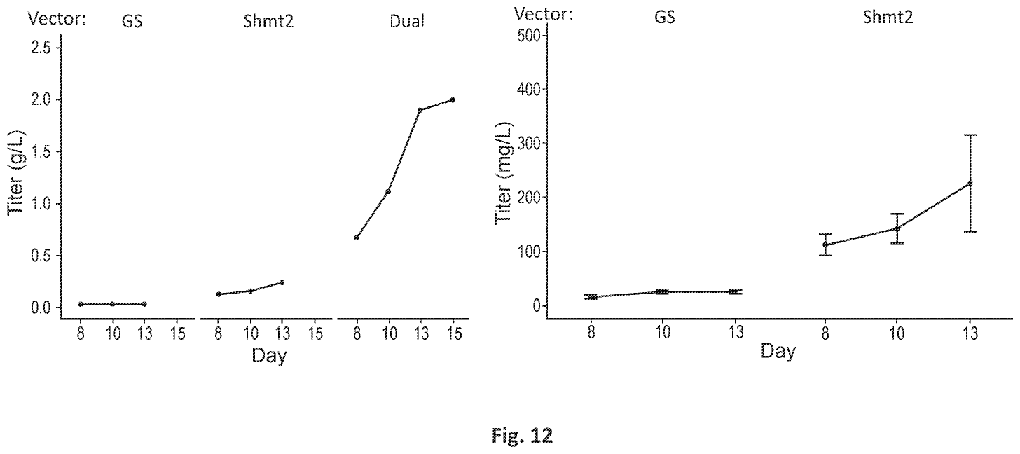

To demonstrate that stable selected cell populations were able to produce a protein of interest using the Gly For-mediated selection system described in Example 1, we developed a vector with an IgG heavy chain, IgG light chain and Serine Hydroxymethyltransferase 2 (SHMT2) coding sequence. This vector was transfected into the CHOZN® GS^{-/-} SHMT2^{-/-} cell line. As a control, mock transfections without DNA were used. The populations had been passaged under selective pressure in Fusion +Gln −Gly −For. The conditions used for selection were also applied during recovery, scale up and during productivity assays. The Fed-batch productivity assay was inoculated at 3e5 viable cells/mL in Advanced +Gln −Gly −For media. Viable cell densities and viabilities for each culture were collected every other day beginning on day 3 post-seeding. Beginning on day 3 post-seeding, 1.5 mL of a 50/50 blend of Advanced Feed −Gly −For and 4 Feed −Gly −For were added to each culture. Glucose readings were taken every other day beginning on day 5 with D-+-Glucose (MilliporeSigma G8769) being added to maintain an appropriate level of glucose. Cells transfected with only the SHMT2 vector reached a peak viable cell density of ˜12.5e6 cells/mL (FIG. 11 left panel middle graph) and maintained >70% viability for at least 13 days (FIG. 11 right panel middle graph). Titers of cells from the fed batch reached a peak of ˜200 mg/L (FIG. 12 left panel middle graph and right panel right graph).

### Example 5

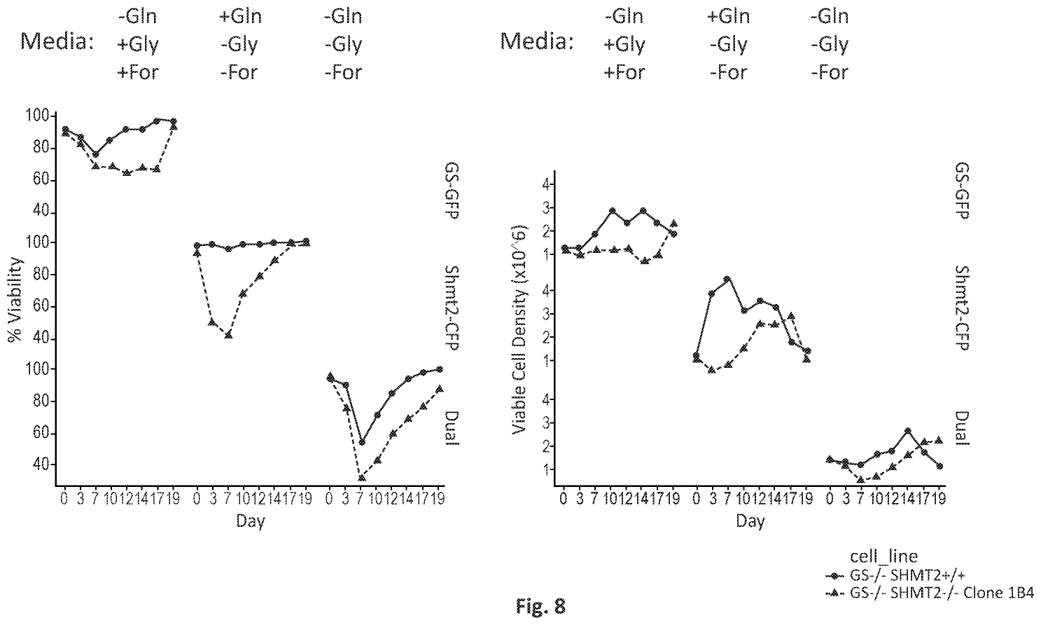

To test if stable cell populations would be able to produce two independent intracellular florescent proteins under glutamine- and glycine/formate-free conditions, we developed two vectors, one containing a GFP and GS coding sequence and a second containing an CFP and SHMT2 coding sequence (FIG. 4). These two plasmids were co-transfected into CHOZN® GS^{-/-} SHMT2^{-/-} cells (GFP+CFP). As controls, each vector was also transfected independently into CHOZN® GS^{-/-} SHMT2^{-/-} cells (GFP only and CFP only, respectively). Cells from all three transfections were then passaged under GS-selective conditions (Fusion −Gln), SHMT2-selective conditions (Fusion −Gly −For) as well as dual metabolic selection conditions (Fusion −Gln −Gly −For). The conditions used for selection were also applied during recovery, scale up and all other-assays. FIG. 8 shows viability data from selection assays indicating that cells transfected with the GFP vector can survive and grow in −Gln conditions but require Gly/For supplementation in the media. On the other hand, cells transfected with the CFP vector can survive and grow in −Gly conditions but require Gln supplementation in the media. Cells co-transfected with both vectors (GFP+CFP) can survive and grow in −Gln −Gly −For media. FIGS. 6 and 8 indicate that cells transfected with the GFP vector that survive and grow in −Gln media are positive for GFP, cells transfected with the CFP vector that survive and grow in −Gly −Formate media are positive for CFP and cells co-transfected with both vectors (GFP+CFP) that survive and grow in −Gln −Gly −For media, are positive for both GFP and CFP. This data indicates that the GS+SHMT2 dual metabolic selection system offers the unique opportunity to select cells in which multiple independent vectors encoding intracellular proteins have been introduced without the need for the addition of any selective agent to the media, for example antibiotics.

### Example 6

To test if stable cell populations would be able to produce secretory proteins under glutamine- and glycine-free conditions, we developed two vectors expressing an IgG1, one containing an IgG heavy chain, IgG light chain and GS coding sequence and a second containing the same IgG heavy chain, IgG light chain and SHMT2 coding sequence. These two independent vectors were co-transfected into CHOZN® GS^{-/-} SHMT2^{-/-} cells (GS+SHMT2). As controls, each vector was also transfected independently into CHOZN® GS^{-/-} SHMT2^{-/-} (GS only and SHMT2 only). Cells were then passaged under selective pressure in Fusion −Gln (GS only transfected cells), Fusion −Gly −For (SHMT2 only transfected cells) or Fusion −Gln −Gly −For (GS+SHMT2 transfected cells). The conditions used for selection were also applied during recovery, scale up and during productivity assays. GS only selection cultures fully recovered after 14-19 days, while the SHMT2 only and GS+SHMT2 dual selection cultures had similar selection recovery profiles and required 19 days to fully recover. In a fed batch assay GS only and SHMT2 only clones produce similar levels of IgG, while GS+SHMT2 cells produce significantly more IgG and have the highest growth and viability. This suggests that the GS and SHMT2 produced by the cell upon expression of an exogenous GS and/or SHMT2 coding sequence is sufficient to express a secreted protein (FIG. 11 right graphs and FIG. 12 left panel right graph). This offers the potential to run large scale production bioreactors under dual metabolic selective conditions, which might be difficult to perform using an antibiotic selection method, either due to the requirement to later separate or purify the antibiotic from the desired secreted protein, or due to the cost of adding an antibiotic to a large-scale bioreactor. Furthermore, this dual metabolic selection system (GS+SHMT2) offers the opportunity to more efficiently select cells in which multiple large vectors have been introduced into the cell, for example when expressing a bispecific antibody or another large and/or complex protein.

What is claimed is:

1. A method for producing a recombinant protein product, the process comprising
(a) providing a mammalian cell line engineered to have reduced or eliminated expression of endogenous serine hydroxymethyltransferase 2 (SHMT2);
(b) introducing into the mammalian cell line a polynucleotide, wherein the polynucleotide encodes a functional SHMT2 gene and the recombinant protein;
(c) culturing the cell line; and
(d) purifying the recombinant protein to form the recombinant protein product.

2. The method of claim 1, wherein the mammalian cell line of (a) further comprises reduced or eliminated expression of endogenous glutamine synthetase (GS), phosphoserine phosphatase (PSPH), dihydrofolate reductase (DHFR), P5C synthase (P5CS), asparaginase (ASPG), alanine transaminase (ALT) and/or asparagine synthetase (ASNS).

3. The method of claim 1, wherein the endogenous SHMT2 expression is reduced or eliminated by inactivating the endogenous SHMT2 gene of the mammalian cell line.

4. The method of claim 1, wherein the endogenous SHMT2 gene is inactivated using a targeting endonuclease-mediated genome modification technique.

5. The method of claim 4, wherein the targeting endonuclease is a CRISPR ribonucleoprotein complex or a pair of zinc finger nucleases.

6. The method of claim 1, wherein the mammalian cell line is a Chinese hamster ovary (CHO) cell line, a baby hamster kidney (BHK) cell line, a NS0 mouse myeloma cell line, a HEK293 cell line, or a Vero African green monkey kidney cell line.

7. The method of claim 6, wherein the cell line is a CHO cell line cultured with or without glycine and/or formate.

8. The method of claim 1, wherein the recombinant protein product is chosen from an antibody, an antibody fragment, a vaccine, a growth factor, a cytokine, a hormone, or a clotting factor.

9. The method of claim 8, wherein the antibody is a bispecific or multispecific antibody.

10. A genetically engineered mammalian cell line for use in a biologic production system, wherein the mammalian cell line is engineered to have reduced or eliminated expression of endogenous SHMT2.

11. The mammalian cell line of claim 10, wherein expression of SHMT2 is reduced or eliminated via inactivation of at least one allele of a chromosomal sequence encoding SHMT2.

12. The mammalian cell line of claim 11, wherein one or more alleles of the chromosomal sequence encoding SHMT2 are inactivated.

13. The mammalian cell line of claim 10, wherein the cell line is engineered to have reduced or eliminated expression of endogenous glutamine synthetase (GS), phosphoserine phosphatase (PSPH), dihydrofolate reductase (DHFR), P5C synthase (P5CS), asparaginase (ASPG), alanine transaminase (ALT) and/or asparagine synthetase (ASNS).

14. The mammalian cell line of claim 12, wherein the chromosomal sequence is inactivated using a targeting endonuclease-mediated genome modification technique.

15. The mammalian cell line of claim 14, wherein the targeting endonuclease is a ribonucleoprotein complex or a pair of zinc finger nucleases.

16. The mammalian cell line of claim 15, wherein the non-human cell line is a Chinese hamster ovary (CHO) cell line, a baby hamster kidney (BHK) cell line, a NS0 mouse myeloma cell line, a HEK293 cell line, or a Vero African green monkey kidney cell line.

17. The mammalian cell line of claim 16 wherein the cell line is a CHO cell line cultured with or without glycine and/or formate.

18. The mammalian cell line of claim 17, in which cell viability, viable cell density, titer, growth rate, proliferation response, cell morphology, and/or general cell health are comparable to those of a non-engineered parental mammalian cell line.

19. The mammalian cell line claim 10, further comprising at least one nucleic acid encoding a recombinant protein chosen from an antibody, an antibody fragment, a vaccine, a growth factor, a cytokine, a hormone, or a clotting factor.

20. The mammalian cell line of claim 19, wherein the antibody is a bispecific or multispecific antibody.

21. A polynucleotide comprising a nucleic acid sequence encoding a functional SHMT2 and at least one recombinant protein of interest.

22. A polynucleotide comprising
a) a nucleic acid sequence encoding a functional SHMT2;
b) a nucleic acid sequence encoding a functional GS and/or ASNS; and
c) a nucleic acid sequence encoding a mutation in the ASNS and/or SHMT2 coding sequence that attenuates the activity of either one or both of the enzymes
d) a nucleic acid sequence encoding a recombinant protein of interest.


SEQUENCE LISTING (38 sequences)

SEQ ID NO: 1 - protein, 7 aa, synthetic construct
PKKKRKV

SEQ ID NO: 2 - protein, 7 aa, synthetic construct
PKKKRRV

SEQ ID NO: 3 - protein, 16 aa, synthetic construct
KRPAATKKAGQAKKKK

SEQ ID NO: 4 - protein, 11 aa, synthetic construct
YGRKKRRQRRR

SEQ ID NO: 5 - protein, 9 aa, synthetic construct
RKKRRQRRR

SEQ ID NO: 6 - protein, 9 aa, synthetic construct
PAAKRVKLD

SEQ ID NO: 7 - protein, 11 aa, synthetic construct
RQRRNELKRSP

SEQ ID NO: 8 - protein, 8 aa, synthetic construct
VSRKRPRP

SEQ ID NO: 9 - protein, 8 aa, synthetic construct
PPKKARED

SEQ ID NO: 10 - protein, 8 aa, synthetic construct
PQPKKKPL

SEQ ID NO: 11 - protein, 12 aa, synthetic construct
SALIKKKKKMAP

SEQ ID NO: 12 - protein, 7 aa, synthetic construct
PKQKKRK

SEQ ID NO: 13 - protein, 10 aa, synthetic construct
RKLKKKIKKL

SEQ ID NO: 14 - protein, 10 aa, synthetic construct
REKKKFLKRR

SEQ ID NO: 15 - protein, 20 aa, synthetic construct
KRKGDEVDGVDEVAKKKSKK

SEQ ID NO: 16 - protein, 17 aa, synthetic construct
RKCLQAGMNLEARKTKK

SEQ ID NO: 17 - protein, 38 aa, synthetic construct
NQSSNFGPMKGGNFGGRSSGPYGGGGQYFAKPRNQGGY

SEQ ID NO: 18 - protein, 42 aa, synthetic construct
RMRIZFKNKGKDTAELRRRRVEVSVELRKAKKDEQILKRRNV

SEQ ID NO: 19 - protein, 20 aa, synthetic construct
GRKKRRQRRRPPQPKKKRKV

SEQ ID NO: 20 - protein, 19 aa, synthetic construct
PLSSIFSRIGDPPKKKRKV

SEQ ID NO: 21 - protein, 24 aa, synthetic construct
GALFLGWLGAAGSTMGAPKKKRKV

SEQ ID NO: 22 - protein, 27 aa, synthetic construct
GALFLGFLGAAGSTMGAWSQPKKKRKV

SEQ ID NO: 23 - protein, 21 aa, synthetic construct
KETWWETWWTEWSQPKKKRKV

SEQ ID NO: 24 - protein, 11 aa, synthetic construct
YARAAARQARA

SEQ ID NO: 25 - protein, 11 aa, synthetic construct
THRLPRRRRRR

SEQ ID NO: 26 - protein, 11 aa, synthetic construct
GGRRARRRRRR

SEQ ID NO: 27 - protein, 12 aa, synthetic construct
RRQRRTSKLMKR

SEQ ID NO: 28 - protein, 27 aa, synthetic construct
GWTLNSAGYLLGKINLKALAALAKKIL

SEQ ID NO: 29 - protein, 33 aa, synthetic construct
KALAWEAKLAKALAKALAKHLAKALAKALKCEA

SEQ ID NO: 30 - protein, 16 aa, synthetic construct
RQIKIWFQNRRMKWKK

SEQ ID NO: 31 - DNA, 1515 bp, unidentified
  source 1..1515: mol_type = unassigned DNA
atggtacccttctctttgctttggaccactcggcctctgcagagatgtggtcagctggtctggatggctgctcgggctcagcacagtaaggtggcccggacacaggctggggaagcagctggaggctggacaggccaggagactttggcagatagtgaccctgagatgtgggagctactgcagagagagaaggacaggcagtgtcgtggcctggagctcatcgcctcagagaacttctgcagccgagctgcgctggaggccctggggtcctgtctgaacaacaagtactcggaaggttaccctggcaagagatattatggaggagcagaagttgtggatgaaatcgagttgctctgccagcgccgggccttggaagcctttgacctggaccctgcacaatggggagtcaatgtgcagccatactctgggtctccagccaatctggctgcttacacagctcttcttcagccccatgatcgaatcatggggttggacctacctgatgggggccacctcactcatggctacatgtctgatgtcagacggatctctgccacatccatcttctttgaatctatgccctataaactcaacccccaaactggcctcattgactacgaccagctggcgttgactgctcggcttttccgaccacggctcatcatagctggtactagtgcctacgcccgtctcattgactatgcccgcatgagagaggtgtgtgatgaggtcaaggcacacctcctggcagacatggcccatatcagtggccttgtggctgccaaggtgatcccgtcacctttcaagtatgcggacattgtcaccaccaccactcacaagactcttcgaggagccaggtcaggactcatcttttatcggaagggagtgcggaccatagaccccaagactggccaagagatcccttacacatttgaggaccgaatcaactttgctgtgttcccatccttacaggggggcccccacaaccatgccattgctgcagtagctgtggccctcaagcaggcctgcacccccatgttccgggagtatgccctacaggtgctgaggaatgctcaggccatggctgatgccctgctaaagagaggctactcactggtgtctggtggcactgacacccacctggtgctggtggacctgcgacccaagggcctggatggagcccgagctgagcgggtgttggagcttgtgtccatcacggccaacaagaacacctgtcctggagaccggagtgccattactcccggaggcctgaggcttggtgccccagcactgacttctcggcagttccgtgaggatgacttccgtagggttgttgattttatcgatgaaggagtcaacattggcttggaggtgaagcgcaagactgccaagctccaggatttcaaatccttcctgctcaaagatgcagaaacaagtcaacgtttggccaacctcaggcaacaagtggagcagtttgccagggccttccccatgcctgggtttgatgaacgctga

SEQ ID NO: 32 - DNA, 107 bp, synthetic construct
  source 1..107: mol_type = other DNA
gtgcagccatactctgggtctccagccaatctggctgcttacacagctcttcttcagccccatgatcgaatcatggggttggacctacctgatgggggccagtgagt

SEQ ID NO: 33 - protein, 34 aa, synthetic construct
VQPYSGSPANLAAYTALLQPHDRIMGLDLPDGGH

SEQ ID NO: 34 - DNA, 85 bp, synthetic construct
  source 1..85: mol_type = other DNA
gtgcagccatactctgggtctccagccaatctggctgcttacacagctcaggtaggttggacctacctgatgggggccagtgagt

SEQ ID NO: 35 - DNA, 103 bp, synthetic construct
  source 1..103: mol_type = other DNA
gtgcagccatactctgggtctccagccaatctggctgcttacacagctcttcttcagccccatgatcgatggggttggacctacctgatgggggccagtgagt

SEQ ID NO: 36 - DNA, 106 bp, synthetic construct
  source 1..106: mol_type = other DNA
gtgcagccatactctgggtctccagccaatctggctgcttacacagctcttcttcagccccatgatcgatcatggggttggacctacctgatgggggccagtgagt

SEQ ID NO: 37 - DNA, 108 bp, synthetic construct
  source 1..108: mol_type = other DNA
gtgcagccatactctgggtctccagccaatctggctgcttacacagctcttcttcagccccatgatcgaaatcatggggttggacctacctgatgggggccagtgagt

SEQ ID NO: 38 - DNA, 102 bp, synthetic construct
  source 1..102: mol_type = other DNA
gtgcagccatactctgggtctccagccaatctggctgcttacacagctcttcttcagccccatgatcatggggttggacctacctgatgggggccagtgagt